(19) United States
(12) Patent Application Publication
(10) Pub. No.: US 2026/0133187 A1
(43) Pub. Date: May 14, 2026

(54) CMD Linkers for Label-Free Molecular Analysis, Methods of Use and Manufacture

(71) Applicant: Nicoya Lifesciences Inc., Kitchener (CA)

(72) Inventors: Hojjat SEYED JAMALI, New Hamburg (CA); Azadeh PIRI SEDIGH, New Hamburg (CA); Terese DIMECK, Kitchener (CA); Kyle PICCOLO, Kitchener (CA)

(21) Appl. No.: 19/386,042

(22) Filed: Nov. 11, 2025

Related U.S. Application Data
(60) Provisional application No. 63/719,105, filed on Nov. 11, 2024.

(51) Int. Cl. G01N 33/543

(52) U.S. Cl. CPC G01N 33/54373; G01N 33/54346; G01N 2400/22

(57) ABSTRACT

Disclosed herein are compositions, sensors, and kits for a carboxymethyl dextran (CMD) based linker for label-free molecular analysis. Further disclosed are methods for synthesizing a CMD-based linker for label-free molecular analysis and method for using reagent kits to immobilize a ligand on a sensor including a CMD-based linker for label-free molecular analysis.

### CROSS REFERENCE TO RELATED APPLICATIONS

This application claims benefit of priority to U.S. Application Ser. No. 63/719,105 filed Nov. 11, 2024, the contents of which is incorporated in its entirety herein.

### TECHNICAL FIELD

The presently disclosed subject matter relates generally to dextran-based linkers for label-free molecular detection of molecules, such as DNA, proteins, and the like, and more particularly to the binding and analysis of analytes using nanostructures and surface plasmon resonance with novel dextran-based linkers for improved molecular detection.

### BACKGROUND

Surface plasmon resonance (SPR) is a label-free analysis technique that can be used to probe molecular binding interactions by detecting a change in the refractive index at the surface caused by binding an analyte of interest. In SPR, a ligand serves as a capture molecule capable of capturing an analyte from solution and may be anchored to an SPR sensor via a linker. The SPR sensor may consist of a metal thin film, metal nanostructures, or metal nanoparticles which are deposited onto a substrate. Detection of ligand-analyte interactions using SPR is accomplished by immobilizing a ligand to a linker connected to the sensor surface and capturing an analyte of interest via the ligand using high-affinity interactions such as antibody capture, streptavidin/biotin, protein-protein interaction, or DNA oligomer counterparts interactions to directly observe the binding of the immobilized ligand to the analyte of interest.

To detect an analyte, a carrier fluid comprising an analyte of interest is passed over the SPR sensor. When the analyte binds to the ligand connected to the sensor, the change in the SPR signal can be quantified as a shift in the position of the SPR absorbance peak as measured by optical absorbance spectroscopy. The SPR peak shift is dependent on the difference between the refractive index of the SPR sensor with bound analyte as compared to the background refractive index of the sensor without bound analyte in the presence of the carrier fluid. The change in refractive index can be interpreted as the change in mass of molecules within the plasmon decay length (or evanescent field) of the sensor and can therefore be used to understand molecular binding reactions between the ligand immobilized proximal to the surface and the analyte of interest in solution that interacts with the ligand. The peak shift is proportional to the amount of analyte bound to the immobilized ligand. Accordingly, an increase in the amount of analyte bound to a ligand immobilized on the sensor surface causes a greater peak shift.

SPR can be used to study, for example, molecular interactions kinetics, binding affinity, analyte concentration, binding specificity, thermodynamic analysis of molecular binding and small molecule interactions. SPR has also been used in various research areas, for example in clinical diagnosis, biomolecular research, environmental monitoring, antigen and antibody detection, chemical detection, food safety, and agricultural research. Various analytes of interest can be detected using SPR including, for example, biological molecules such as nucleic acids, amino acids, proteins, peptides, oligomers, DNA, RNA, cells, and dissolved organic molecules. Optical sensing techniques using light transmission and SPR can also be used in combination with digital microfluidics devices and systems for the optical measurement of the presence of and concentration of dissolved and dispersed components in microfluidic droplets.

Many SPR applications use a self-assembled monolayer (SAM) composed of a plurality of linkers. Linkers for use in SPR generally have an anchor end for binding to the sensor surface, an attachment group for coupling to a ligand, and a linker therebetween. Deposition of vertically oriented linkers on the sensor surface can be challenging because of the flexibility of the linker and the affinity of the surface to the linker, which typically includes carboxylic acid groups, which can lead to randomness in the orientation of the linker on the sensor surface. Linkers of various types having various kinds of binding groups have been used in SPR and are generally constructed piecewise via bottom-up self-assembly onto the sensor surface. In one example, Tabasi et al. (Applied Surface Science 490 (2019) 251-259) describe a multi-step synthesis for assembling a linker on an Ag—Au bimetallic film and attaching an antibody ligand to the end of the linker.

In many applications in which SPR is used to measure binding kinetics, the analyte being measured has a relatively small molecular weight. Because the SPR signal shift depends on the change in mass close to the sensor surface, the larger the molecular weight of the analyte bound to the ligand the larger the shift in the refractive index of the sensor. As such, a small molecule analyte may not produce a distinct change in refractive index during binding and this can lead to difficulty in accurately measuring binding kinetics, specificity, affinity, etc. Compared to SPR, localized surface plasmon resonance (LSPR) provides higher sensitivity, however, the integration of the LSPR advantages with the aforementioned linker design is challenging because this type of linker is generally built on top of the plasmonic surface through a stepwise in situ process that might damage the sensor configuration due to the fragile structure and/or adhesion of metal nanoparticles and the harsh conditions required for the synthesis.

This background information is provided for the purpose of making known information believed by the applicant to be of possible relevance to the present invention. No admission is necessarily intended, nor should be construed, that any of the preceding information constitutes prior art against the present invention.

### BRIEF SUMMARY OF THE INVENTION

Disclosed herein are compositions, sensors, and reagent kits for a carboxymethyl dextran (CMD) based linker for label-free molecular analysis. Further disclosed are methods for synthesizing a CMD-based linker for label-free molecular analysis and methods for using reagent kits to immobilize a ligand on a sensor including a CMD-based linker for label-free molecular analysis.

In one aspect, disclosed herein is a composition for label-free molecular analysis, including a compound of Formula I:

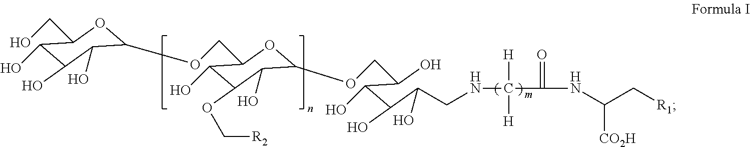

where R₁ includes sulfur, carbon, nitrogen, oxygen, selenium, tellurium, or polonium; R₂ includes carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, N-hydroxysuccinimide ester, nitrilotriacetic acid, azide, alkyne, a photocleavable moiety, or a photoreactive moiety; m is about 1 to about 6000; and n is about 1 to about 80.

In some cases, R₁ includes sulfur and may include thiol, sulfide, disulfide, or any combinations thereof. In some cases, wherein R₁ includes nitrogen and may include diazonium salt, amine, amino acid, or any combinations thereof. In some cases, R₁ includes an amine and may include a primary amine, a secondary amine, a tertiary amine, a quaternary amine, or any combinations thereof. In some cases, R₁ includes a thiol. In some cases, R₂ includes carboxyl.

In some cases, m is about 1 to about 3000. In some cases, m is 37. In some cases, n is about 1 to about 30. In some cases, n is 10.

In one aspect, the composition of Formula I may include: R₁ includes a thiol; R₂ includes carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, N-hydroxysuccinimide ester, nitrilotriacetic acid, azide, alkyne, a photocleavable moiety, or a photoreactive moiety; m is about 1 to about 6000; and n is about 1 to about 80.

In one aspect, the composition of Formula I may include: R₁ includes sulfur, carbon, nitrogen, oxygen, selenium, tellurium, or polonium; R₂ includes carboxyl; m is about 1 to about 6000; and n is about 1 to about 80.

In another aspect, the composition of Formula I may include: R₁ includes sulfur, carbon, nitrogen, oxygen, selenium, tellurium, or polonium; R₂ includes carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, N-hydroxysuccinimide ester, nitrilotriacetic acid, azide, alkyne, a photocleavable moiety, or a photoreactive moiety; m is 37; and n is about 1 to about 80.

In one aspect, the composition of Formula I may include: R₁ includes sulfur, carbon, nitrogen, oxygen, selenium, tellurium, or polonium; R₂ includes carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, N-hydroxysuccinimide ester, nitrilotriacetic acid, azide, alkyne, a photocleavable moiety, or a photoreactive moiety; m is about 1 to about 6000; and n is 10.

In one aspect, the compound of Formula I may include: R₁ includes thiol; R₂ includes carboxyl; m is about 1 to about 6000; and n is about 1 to about 80.

In one aspect, the compound of Formula I may include: R₁ includes thiol; R₂ includes carboxyl; m is about 1 to about 3000; and n is about 1 to about 30.

In one aspect, the compound of Formula I may include: R₁ includes thiol; R₂ includes carboxyl; m is 37; and n is 10.

In another aspect, disclosed herein is a composition for label-free molecular analysis, including a compound of Formula II:

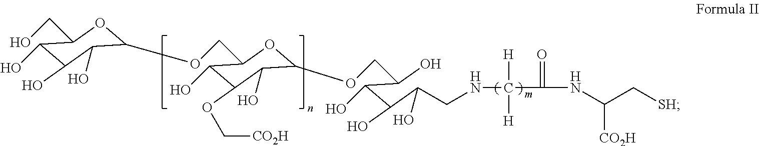

where m is 1 to about 6000; and n is 1 to about 80.

In some cases, m is about 1 to about 6000 and n is about 1 to about 30. In other cases, m is about 1 to about 6000 and n is 10. In some cases, m is about 1 to about 3000 and n is about 1 to about 80. In some cases, m is 37 and n is about 1 to about 80. In one case, m is 37 and n is 10.

Also disclosed is a sensor for label-free molecular analysis. The sensor includes a sensor surface including a substrate coated with a metal layer and a linker disposed on the metal later, the linker including a compound of Formula I:

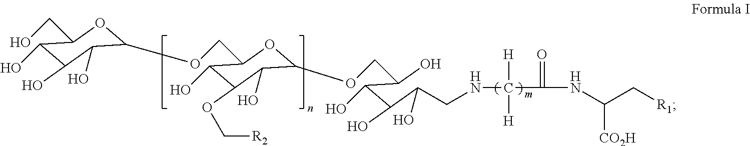

where R₁ includes sulfur, carbon, nitrogen, oxygen, selenium, tellurium, or polonium; R₂ includes carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, N-hydroxysuccinimide ester, nitrilotriacetic acid, azide, alkyne, a photocleavable moiety, or a photoreactive moiety; m is about 1 to about 6000; and n is about 1 to about 80.

In some cases, the sensor surface further includes a polyelectrolyte between the substrate and the metal layer.

In some cases, the substrate includes silica, glass or quartz. In some cases, the sensor substrate includes an optical fiber.

In some cases, the metal layer is a metal film. In some cases, the metal layer is selected from a one or more of gold, silver, platinum, copper, gold coated silver, silver coated gold, a metal-coated nonmetal, metal coated non-metal nanoparticles, a combination of metals or mixtures thereof. In some cases, the metal layer includes a plurality of nanoparticles.

In another aspect, the sensor may include a surface plasmon resonance (SPR) sensor. In some cases the surface plasmon resonance sensor is a localized surface plasmon resonance (LSPR) sensor.

In another aspect, the metal layer of the sensor may include gold nanoparticles and the linker includes a compound of Formula II:

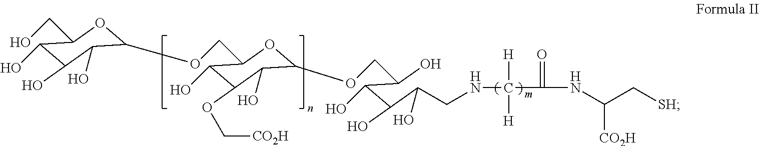

where m is 1 to about 6000; and n is 1 to about 80.

In some cases, m is about 1 to about 3000. In some cases, m is 37. In some cases, n is about 1 to about 30. In some cases, n is 10.

In one aspect, the sensor includes a compound of Formula I where R₁ includes a thiol; R₂ includes carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, N-hydroxysuccinimide ester, nitrilotriacetic acid, azide, alkyne, a photocleavable moiety, or a photoreactive moiety; m is about 1 to about 6000; and n is about 1 to about 80.

In one aspect, the sensor includes a compound of Formula I where R₁ includes sulfur, carbon, nitrogen, oxygen, selenium, tellurium, or polonium; R₂ includes carboxyl; m is about 1 to about 6000; and n is about 1 to about 80.

In one aspect, the sensor includes a compound of Formula I where R₁ includes sulfur, carbon, nitrogen, oxygen, selenium, tellurium, or polonium; R₂ includes carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, N-hydroxysuccinimide ester, nitrilotriacetic acid, azide, alkyne, a photocleavable moiety, or a photoreactive moiety; m is 37; and n is about 1 to about 80.

In one aspect, the sensor includes a compound of Formula I where R₁ includes sulfur, carbon, nitrogen, oxygen, selenium, tellurium, or polonium; R₂ includes carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, N-hydroxysuccinimide ester, nitrilotriacetic acid, azide, alkyne, a photocleavable moiety, or a photoreactive moiety; m is about 1 to about 6000; and n is 10.

In one aspect, the sensor includes a compound of Formula I where R₁ includes thiol; R₂ includes carboxyl; m is about 1 to about 6000; and n is about 1 to about 80.

In one aspect, the sensor includes a compound of Formula I where R₁ includes thiol; R₂ includes carboxyl; m is about 1 to about 3000; and n is about 1 to about 30.

In one aspect, the sensor includes a compound of Formula I where R₁ includes thiol; R₂ includes carboxyl; m is 37; and n is 10.

In another aspect, disclosed herein is a reagent kit for treating a sensor surface having a linker disposed thereon. The linker may include a compound of Formula I:

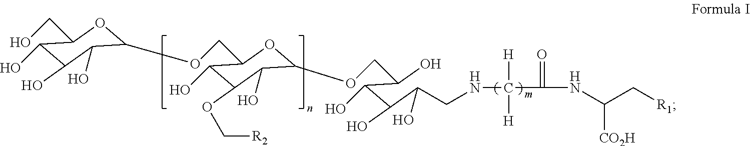

where R₁ includes sulfur, carbon, nitrogen, oxygen, selenium, tellurium, or polonium. R₂ includes carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, N-hydroxysuccinimide ester, nitrilotriacetic acid, azide, alkyne, a photocleavable moiety, or a photoreactive moiety. In some cases, m is about 1 to about 6000, and n is about 1 to about 80. The reagent kit may include a first solution including an acid and a surfactant, a second solution including a ligand, and a third solution including an amine.

In some cases, the linker may include a compound of Formula II:

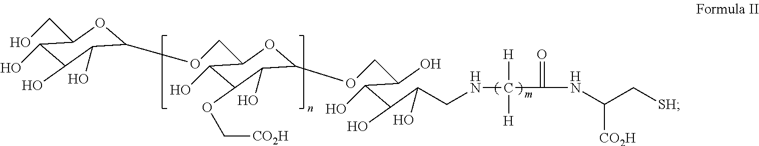

where m is 1 to about 6000; and n is 1 to about 80.

In some cases, m is about 1 to about 3000. In some cases, m is 37. In some cases, n is about 1 to about 30. In some cases, n is 10.

In one aspect, the first solution includes an inorganic acid and a surfactant. In some cases, the first solution includes hydrochloric acid and a non-ionic surfactant. In some cases, the first solution includes from about 0.1 mM to about 1 M hydrochloric acid; and from about 0.01% to about 10% of a non-ionic surfactant, based on the total volume of the solution.

In another aspect, the second solution includes a protein, a peptide, an antibody, aptamer, polymer, DNA, or any combinations thereof. In some cases, the second solution further includes a coupling agent. In some cases, the coupling agent includes 1-Ethyl-3-(3-dimethylaminopropyl)carbodiimide (EDC)/N-hydroxysuccinimide(NHS), EDC/sulfo-N-hydroxysuccinimide, 4-(4,6-Dimethoxy-1,3,5-triazin-2-yl)-4-methylmorpholinium chloride (DMTMM), or combinations thereof.

In another aspect, the third solution includes a primary amine. In some cases, the third solution includes ethanolamine, tris(hydroxymethyl)aminomethane (tris), glycine, lysine, amino alcohols (e.g., 1-amino-2-propanol), PEG-amine derivatives, proteins, or any combinations thereof. In some cases, the third solution includes from about 0.5 M to about 4 M ethanolamine.

Also disclosed herein is a composition for label-free molecular analysis including a compound of Formula III:

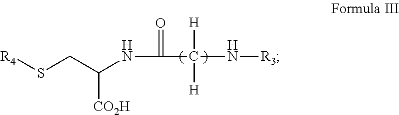

where R₃ includes a monosaccharide, a disaccharide, a polysaccharide, or an oligosaccharide; R₄ includes hydrogen or a protecting group; and m is about 1 to about 6000.

In some cases, R₃ includes triose, pentose, hexose, heptose, or any combinations thereof.

In some cases, R₃ includes glucose, fructose, galactose, ribose, deoxyribose, mannose, and any combinations thereof.

In some cases, R₃ includes sucrose, lactose, maltose, trehalose, cellobiose, chitobiose, kojibiose, nigerose, isomaltose, sophorose, laminaribiose, gentiobiose, turanose, maltulose, leucrose, isomaltulose, gentiobiulose, mannobiose, melibiose, allolactose, lactulose, rutinose, rutinulose, xylobiose, or any combinations thereof.

In some cases, R₃ includes dextran, amylose, glycogen, cellulose, chitin, starch, pectin, hemicellulose, inulin, agar, alginate, or any combinations thereof.

In some cases, R₃ includes fructo-oligosaccharides, galactooligosaccharides, mannan oligosaccharides, or any combinations thereof.

In some cases, R₃ includes a compound of Formula IV.

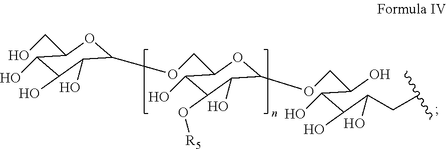

where n is about 1 to about 80; and R₅ includes hydrogen or carboxyl.

In another aspect, the composition for label-free molecular analysis includes a compound of Formula III where R₃ includes dextran; R₄ includes hydrogen or a protecting group; R₅ includes hydrogen or carboxyl; m is about 1 to about 6000; and n is about 1 to about 80.

In another aspect, the composition for label-free molecular analysis includes a compound of Formula III where R₃ includes a monosaccharide, a disaccharide, a polysaccharide, or an oligosaccharide; R₄ includes hydrogen; R₅ includes hydrogen or carboxyl; m is about 1 to about 6000; and n is about 1 to about 80.

In another aspect, the composition for label-free molecular analysis includes a compound of Formula III where R₃ includes a monosaccharide, a disaccharide, a polysaccharide, or an oligosaccharide; R₄ includes H or a protecting group; R₅ includes H or carboxyl; m is about 1 to about 3000; and n is about 1 to about 30.

In another aspect, the composition for label-free molecular analysis includes a compound of Formula III where R₃ includes a monosaccharide, a disaccharide, a polysaccharide, or an oligosaccharide; R₄ includes hydrogen or a protecting group; R₅ includes carboxyl; m is 37; and n is 10.

In another aspect, also disclosed herein is a method for using a reagent kit to immobilize a ligand on a linker-functionalized sensor surface including a compound of Formula I:

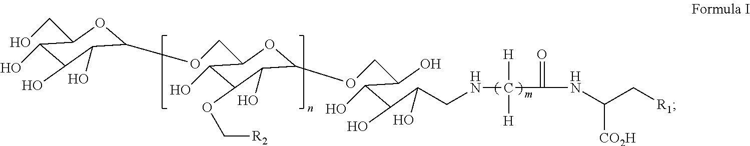

where R₁ includes sulfur, carbon, nitrogen, oxygen, selenium, tellurium, or polonium; R₂ includes carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, N-hydroxysuccinimide ester, nitrilotriacetic acid, azide, alkyne, a photocleavable moiety, or a photoreactive moiety; m is about 1 to about 6000; and n is about 1 to about 80. The method includes: exposing the linker-functionalized sensor surface to a sensor preparation solution including an acid and a surfactant to produce a cleaned linker-functionalized sensor surface; exposing the cleaned linker-functionalized sensor surface to a ligand solution including a ligand to produce a ligand-functionalized sensor surface; and exposing the ligand-functionalized surface to a blocking solution including an amine to produce a quenched ligand-functionalized sensor surface.

In some cases, the method further includes exposing the quenched ligand-functionalized sensor surface to a sensor preparation solution including an acid and a surfactant to produce a cleaned quenched ligand-functionalized sensor surface.

In some cases, also disclosed herein is a method for using a reagent kit to immobilize a ligand on a linker-functionalized sensor surface including a compound of Formula II:

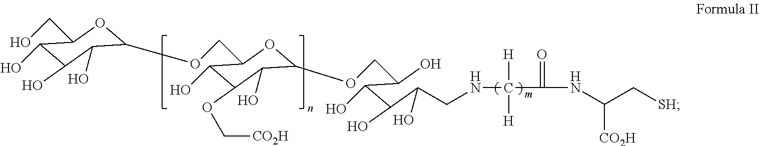

where m is 1 to about 6000; and n is 1 to about 80.

In some cases, the sensor preparation solution includes hydrochloric acid and a non-ionic surfactant. In some cases, the sensor preparation solution includes: from about 0.1 mM to about 1 M hydrochloric acid; and from about 0.01% to about 10% of a non-ionic surfactant, based on the total volume of the solution.

In another aspect, the ligand includes an amine. In some cases, the ligand includes a protein, a peptide, an antibody, an aptamer, a polymer, DNA, or any combinations thereof.

In some cases, the ligand solution further includes a coupling agent. In some cases, the coupling agent includes 1-Ethyl-3-(3-dimethylaminopropyl)carbodiimide (EDC)/N-hydroxysuccinimide(NHS), EDC/sulfo-N-hydroxysuccinimide, 4-(4,6-Dimethoxy-1,3,5-triazin-2-yl)-4-methylmorpholinium chloride (DMTMM), or combinations thereof.

In another aspect, the blocking solution includes ethanolamine, tris(hydroxymethyl)aminomethane (tris), glycine, lysine, amino alcohols (e.g., 1-amino-2-propanol), PEG-amine derivatives, proteins, or any combinations thereof. In some cases, the blocking solution includes about from about 0.5 M to about 4 M ethanolamine.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is an illustration of an exemplary label-free sensor system according to the principles described herein;

FIG. 2A illustrates an exemplary linker for a label-free sensor, according to some embodiments;

FIG. 2B illustrates an exemplary linker for a label-free sensor, according to some embodiments;

FIG. 3 is an illustration of a label-free sensor comprising a self-assembled monolayer consisting of an exemplary linker, according to some embodiments;

FIG. 4 is an illustration of indicating the relationship between plasmon resonance decay length and linker size;

FIG. 5 illustrates an exemplary method, according to some embodiments.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the term “linker” refers to the molecule which is anchored to a metal or a metallic alloy or any other plasmonic material on an SPR sensor surface and provides an attachment site for attachment to a ligand. A collection of a plurality of linkers may form a self-assembled monolayer (SAM).

As used herein, the term “ligand” refers to the chemical moiety that may be coupled to an attachment site on a linker and used to bind to an analyte. The ligand may be, for example, any binder, such as a protein, a peptide, an antibody, aptamer, polymer, DNA or other capture molecule having affinity for an analyte. In some cases the ligand may be present in a solution, referred to as a “ligand solution.”

As used herein, the term “nanoparticle” refers to a particle of varying shapes and sizes ranging from about 1 nm and 1000 nm with a variety of area-to-volume ratios.

As used herein, the term “metal” means any metal capable of producing a surface plasmon (i.e., gold, silver, alloys and composites thereof) and may be either be in the form of a thin film or nanoparticle. Deposition of metal onto the substrate can be done, for example, by sputtering, adhesion, or other method.

The acronym “Cys” refers to the amino acid cysteine.

The acronym “CMD” refers to carboxymethyl dextran.

The acronym “DCM” refers to the solvent dichloromethane.

The acronym “DIPEA” refers to N,N-Diisopropylethylamine, also known as Hunig's base.

The acronym “DMF” refers to the solvent dimethylformamide.

The acronym “DMSO” refers to the solvent dimethylsulfoxide.

The acronym “EDC” refers to the coupling agent 1-Ethyl-3-(3-dimethylaminopropyl)carbodiimide.

The acronym “Fmoc” refers to the fluorenylmethyloxycarbonyl protection group or capping group.

The acronym “HATU” refers to the coupling agent hexafluorophosphate azabenzotriazole tetramethyl uronium.

The acronym “MeOH” refers to the solvent methanol.

The acronym “NHS” refers to the chemical reagent N-Hydroxysuccinimide.

The acronym “TFA” refers to trifluoroacetic acid.

Herein is provided an improved linker for SPR and LSPR for label-free binding. The improved linker is configured to self-assembly on a metal surface of an SPR or LSPR sensor and may comprise at least one attachment site for binding to a ligand. The presently described linker can also be used in methods, devices, sensors, and systems for label-free detection of analytes via SPR, LSPR or other label-free analysis techniques. The use of the presently described linkers improves signal amplification and signal strength for the detection of various analytes, such as proteins, peptides, antibodies, carbohydrates, and nucleic acid molecules. The present linker can be used to provide an increased optical signal shift (or change in refractive index) in plasmon resonance-based techniques. In addition, the present linker may provide multiple ligand attachment sites, thus increasing signal strength and signal-to-noise.

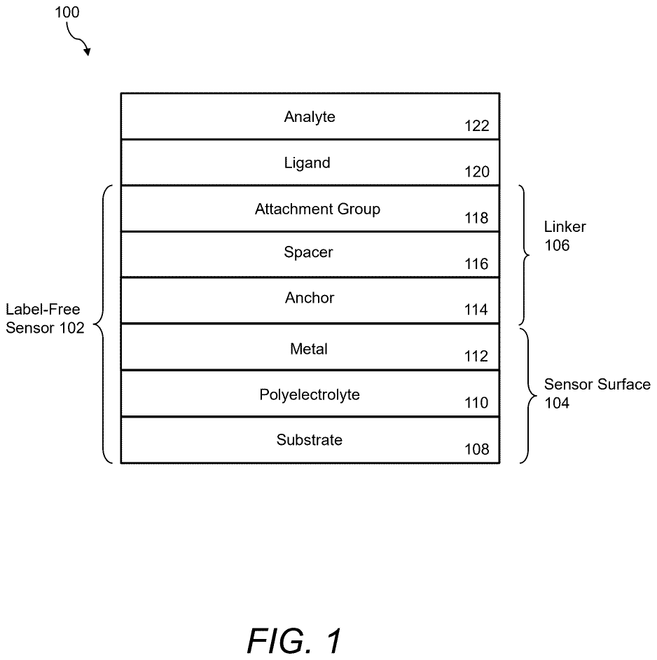

FIG. 1 is an illustration of an exemplary label-free sensor system according to the principles described herein. Sensor system 100 comprises a label-free optical sensor 102 to which a ligand 120 can be attached for investigating the binding of an analyte 122 using various analytical techniques, such as SPR or LSPR techniques. Label-free sensor 102 comprises a sensor surface 104. The sensor surface 104 consists of a substrate 108, such as a silica-based substrate, treated with a polyelectrolyte 110 that is configured to bind a metal 112, such as a plurality of metal nanoparticles. Once bound, the metal 112 is capable of immobilizing a linker 106. The linker 106 comprises an anchor 114 for attachment to the metal 112, connected to a spacer 116 which in turn is connected to at least one attachment group 118 comprising one or more attachment sites for coupling with a ligand 120.

The presently described linker for label-free analysis comprising an anchor for binding to a metal, a spacer connected to the anchor, and at least one attachment group comprising one or more attachment sites for coupling a ligand has numerous advantages compared to known linkers. In the present linker design, there may be multiple attachment sites 118 for coupling to a ligand 120. In this way, the ratio of ligand to linker may be greater than 1, providing more sites for coupling with an analyte of interest thereby increasing signal strength, which is particularly useful in plasmon resonance sensors, such as LSPR sensors. In addition, the presently provided linker may be synthesized as a single entity comprising, in series, the anchor 114, spacer 116, and attachment group 118, which enables self-assembly of the linker on the metal 112 in a single step. In contrast, many current label-free linkers are fabricated in situ by bottom-up self-assembly (i.e., each component of the linker is added to the sensor one at a time). Providing the linker as a single structure enables a one-step preparation for forming a monolayer of the linker onto the sensor surface 104. Additionally, the presence of both hydrophobic or hydrophilic portions of the linker promotes self-assembly of the linker on the metal 112, providing consistency and order throughout the monolayer. Improved ordering of the linker 106 on the sensor surface 104 may provide improved sensor sensitivity and decrease non-specific binding.

Once the linker 106 is assembled onto the sensor surface 104, application of the ligand 118 to the sensor 106 is routine. To do this, a carrier fluid consisting of water or a buffered solution comprising the ligand 120 is contacted with the label-free sensor 102 until the sensor provides a response or signal above a desired threshold.

### CMD Linkers

In one aspect, the present disclosure provides an improved carboxymethyl dextran (CMD) linker for a label-free molecular analysis sensor and system. In some embodiments, the CMD linker comprises the structure of Formula I:

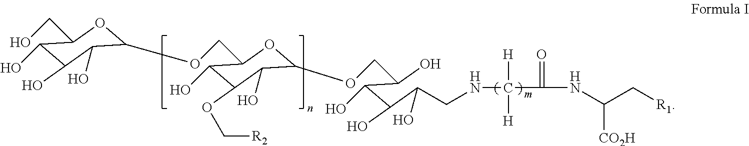

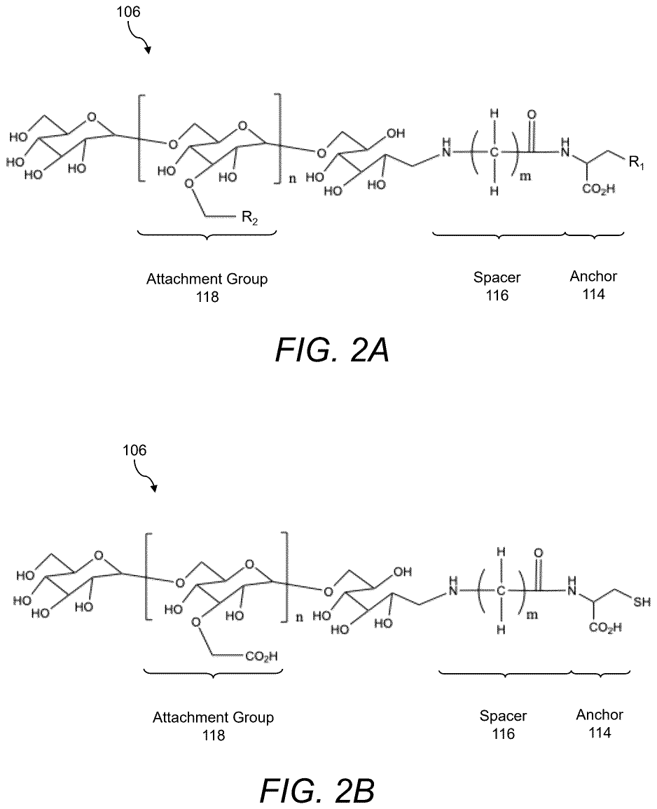

As shown in FIG. 2A, and in Formula I, the improved CMD linker may comprise at least one anchor 112 (R₁), at least one spacer 114, and at least one attachment group 116 comprising one or more attachment sites (R₂). In some cases, R₁ may include sulfur, carbon, nitrogen, oxygen, selenium, tellurium, or polonium. In some cases, R₂ may include carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, NHS-ester, nitrilotriacetic acid (NTA), azide, alkyne, a photocleavable moiety, or a photoreactive moiety. In some embodiments, “n” defines the number of repeat units in attachment group 116 which may be any whole-digit integer from 1 to 80 or from 1 to 30, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 repeat units. Still in some embodiments, “m” defines the number of repeat units in spacer 114 which may be any whole-digit integer from 1 to 6000. In some cases, m may be any whole digit integer from 1 to 3000. In some cases, m includes 1, 2, 3, 4, 5, 6, 7, 8, 9, 10, 11, 12, 13, 14, 15, 16, 17, 18, 18, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 repeat units. In some cases, m is 37. In some cases, n is 10.

In some embodiments, the CMD linker comprises the structure of Formula II:

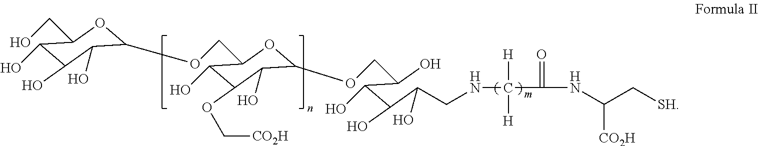

As shown in FIG. 2B, and in Formula II, the improved CMD linker may comprise at least one anchor 112, at least one spacer 114, and at least one attachment group 116 comprising one or more attachment sites. As shown in Formula II, the anchor, R₁, may comprise a thiol and the one or more attachment sites, R₂, may comprise a carboxyl. In some embodiments, “n” defines the number of repeat units in attachment group 116 which may be any whole-digit integer from 1 to 80 or from 1 to 30, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 repeat units. Still in some embodiments, “m” defines the number of repeat units in spacer 114 which may be any whole-digit integer from 1 to 6000. In some cases, m may be any whole digit integer from 1 to 3000. In some cases, m includes 1, 2, 3, 4, 5, 6, 7, 8, 9, 10, 11, 12, 13, 14, 15, 16, 17, 18, 18, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 repeat units. In some cases, m is 37. In some cases, n is 10.

### Anchors

In one aspect, the CMD linker comprises an anchor (R₁ in Formula I). In some embodiments, the anchor may comprise sulfur. In some embodiments, the anchor 114 may be a thiol (—SH) group that immobilizes the linker to the metal on the sensor surface. Shown is a single thiol group on a cysteine amino acid, however, the linker may comprise any molecule having one or more thiol groups or other suitable chemical functional groups capable of binding a metal. Chemisorption of the anchor 114 to the metal 112, such as a metal thin film or metal nanoparticle, provides a simple and efficient mechanism for coupling the linker 106 to the metal 112. A single anchor 114 per linker 106 can provide alignment of the ligand relative to the metal such that the linker extends away from the metal, forming a self-assembled monolayer (SAM). In addition, the regio-chemistry, which refers to the location of the anchor relative to the linker structure, plays a critical role in the final arrangement of the linker 106 on the metal 112. In particular, disposing anchor 114 at the proximal end (i.e., the end closest to the metal) of the linker 106 allows each of linker 106 to align due to similar chemical properties of the components therein forming the SAM. Disposing anchor 114 at a proximal end of linker 106 not only provides for a better arrangement of linker 106 on metal 112 but also prevents the linker from creating loops or bends which would otherwise alter the availability or accessibility of the attachment sites of attachment group 118.

In some embodiments, the anchor 114 comprises one or more moieties capable of binding the linker 106 to the metal 112. In some embodiments, R₁ may include a sulfur moiety, a carbon moiety (e.g., alkyl), a nitrogen moiety, a group 16 (6A) chalcogen moiety, a thiol moiety, a sulfide moiety, a disulfide moiety, a diazonium salt moiety, an amine moiety, an amino acid moiety, or any combination thereof. In one embodiment, R₁ may include an amine moiety and wherein the amine moiety comprises a primary, secondary, tertiary or quaternary amine. In another embodiment, R₁ may include a sulfur moiety and wherein the sulfur moiety comprises a thioacid moiety, a thioacid anion moiety, a carbothioic acid moiety, a carbothioic acid anion moiety, a thiocarboxylic acid moiety, a thiocarboxylic anion moiety or any combination thereof. In still another embodiment, R₁ may include a disulfide moiety and wherein the disulfide moiety comprises a dithiocarbamate moiety or a dithiocarbamic acid moiety or any combination thereof. In yet another embodiment, R₁ may include a chalcogen moiety and wherein the chalcogen moiety comprises Te or Se or a combination thereof.

### Spacers

Attached to the anchor group is a spacer 116. In some embodiments, the spacer 116 may be a hydrophobic spacer comprising a plurality of hydrocarbon groups that may promote self-assembly of the linker through hydrophobic interactions among spacers of adjacent linkers as in some cases the formation of a self-assembled monolayer plays a critical role in the linker arrangement on the metal 110. The spacer 116 can be a hydrophobic portion comprising straight-chained, branched, saturated, or unsaturated hydrocarbons, or optionally have other hydrophilic functionalization thereon. In its simplest form, the spacer 116 can be a methylene linker having from about 1 to about 44 methylene groups. In some embodiments, the spacer 116 may have a molecular weight from about 14 grams per mole (g/mol) (i.e., CH₂) to 1000 g/mol. Still in other embodiments, the spacer 116 may have a length from about 0.1 nanometers (nm) to about 5 nm. Additionally, the spacer 116 may be unsaturated or have unsaturated or aromatic groups therein or thereon to provide additional rigidity to the linker.

### Attachment Groups

Various hydrophilic polysaccharides have been used to prepare linkers for label-free analysis. In some embodiments, the attachment group 118 may be a polysaccharide comprising dextran, aliginate, pullulan, levan, inulin, starch, chitosan, curdlan, xanthan gum, gellan gum and glycogen. In one general class, dextran polysaccharides have been used and can be carboxymethylated to provide attachment sites for coupling to a ligand. In plasmon resonance-based techniques dextran-based linkers have been developed to address several challenges simultaneously. One challenge is reducing non-specific binding (NSB) using hydrophobic domains or portions. Another challenge is providing the possibility of multi-layer immobilization to increase signal strength for small molecule detection. In the present disclosure, the alignment of the hydrophilic portions during self-assembly of the linker on the sensor surface 104 forms a hydrophilic layer, and similarly, the alignment of the hydrophobic portions forms a hydrophilic layer relative to the metal 110, reducing non-specific binding of the analyte 122 to the metal 112 or the linker 106.

The attachment group 118 may comprise one or more attachment sites, indicated by R₂, for immobilizing a ligand to the label-free sensor 102. Various attachment site chemistries can be used, including attachment sites including biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, NHS-ester, or nitrilotriacetic acid (NTA). In some cases, the attachment site may employ ‘Click’ chemistry, or similar cycloadditions, involving attachment sites including azide and/or alkyne functional groups for use in Copper(I)-catalyzed azide-alkyne cycloaddition (CuAAC), Strain-promoted azide-alkyne cycloaddition (SPAAC), or Strain-promoted alkyne-nitrone cycloaddition (SPANC). In some cases, the attachment site may include a photocleavable linker. In some cases, the attachment site may include a photoreactive moiety.

In some cases, the attachment site may include an organic acid, such as carboxyl (i.e., carboxylic acid groups). In some instances, there are approximately 37 repeat units in the structure of CMD with roughly 80% having a carboxyl group for binding a ligand, therefore there may be as many as 30 carboxyl groups per linker chain.

Reactive carboxyl groups can be provided on the attachment group 118 by, for example, carboxy methylation of a polysaccharide molecule to convert some free hydroxyl groups into carboxylic acid groups. These carboxylic acids can then be functionalized with a coupling agent before exposure to a ligand. For example, EDC/NHS coupling may be used to covalently link the carboxylic acid groups of the attachment group to an amine of a ligand. Other immobilization chemistries may also be used to bind a ligand to an attachment site of the attachment group. For example, streptavidin-biotin coupling may be used.

In particular, the conversion of multiple hydroxyl groups on dextran into carboxyl groups and activation of the carboxyl groups for coupling with ligand 120 provides multiple sites for binding analyte 122, and improved signal intensity. In addition, as compared with carbohydrate macrostructures in other sensor designs, the ligand binding sites of the linkers described herein are provided at a specific distance from the sensor surface 106 due to the linker composition and ability to self-assemble on the sensor surface 106.

The CMD-based as presently described may be provided as standalone linkers for functionalization of a sensor surface or may be provided pre-bound to sensors for incorporation into label-free analysis system. Suitable ligands or coupling agents can also be provided as reagent kits for preparing sensors for label-free analysis.

### CMD-Based Label-Free Sensors

The sensors disclosed herein may operate using surface plasmon resonance (SPR) or localized surface plasmon resonance (LSPR). LSPR is a phenomenon associated with noble metal nanoparticles that creates sharp spectral absorbance and scattering peaks and produces strong electromagnetic near-field enhancements. These spectral peaks may be monitored using spectrochemical analysis. The spectral peak changes with refractive index changes in the immediate vicinity of the nanoparticle surface. When chemical targets (analytes of interest) are bound near the surface of a metal nanoparticle, a shift in the spectral peak occurs due to changes in the local refractive index. This may be used to determine the concentration of a specific target in a complex medium. Alternatively, the spectral peak shift may be detected through a change in absorption at a given wavelength.

A sample of interest may be introduced to metal nanoparticles of the sensor, whereby target analytes in the sample bind to their respective capture molecules (i.e., ligands) that are coupled to the surface of the metal nanoparticles by linkers. The overall spectral peak of the sensor shifts according to the concentration of the target analyte proximal to the surface of the metal nanoparticles. In some examples, LSPR sensors with nanoparticles on planar surfaces operate by flowing the sample longitudinally over the surface. In other examples, individual fluid droplets may be introduced to the LSPR sensor by electrowetting on a microfluidic device, such as a digital microfluidic (DMF) device. In order to measure this shift, reflectance or transmission absorbance spectroscopy may be employed. Further, analysis via “intensity or colorimetric methods” may be performed using LSPR sensors.

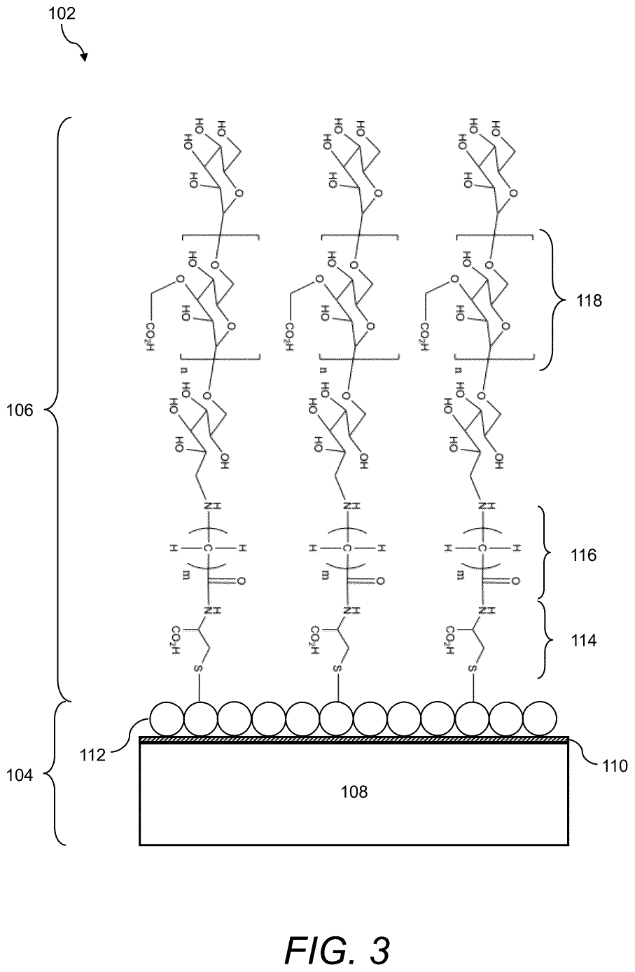

Referring now to FIG. 3 is an illustration of a CMD-based linker self-assembled on a sensor surface 104 of a label-free sensor 102. In some embodiments, the label-free sensor may comprise a substrate 108. In some cases, the substrate is a flat surface or a microstructured surface. In some cases the substrate is a substantially transparent or opaque substrate. That is, substrate may be substantially transparent when used in a transmission mode configuration. By contrast, substrate may be opaque when used in a reflection mode configuration. In some cases, the substrate contains silica, plastic, or TPE. In some cases, the substrate is a glass substrate. In some cases, the substrate is the end of an optical fiber. In some cases, the sensor may be configured as a reflective interferometric sensor such as a bio-layer interferometry (BLI) or single color reflectometry (SCORE). These interferometric sensors are deposited using techniques such as chemical vapor deposition (CVD) or physical vapor deposition (PVD) onto the substrate with typical materials used for thin film coatings such as Zinc Sulfide, Titanium Dioxide, Magnesium Fluoride, Silicon Dioxide with an ultimate metallic layer of plasmonic metals such as gold. In these reflective interferometric sensors, the presence or absence of a target analyte interacting with the ligand or capture molecule will cause a wavelength shift or change in interference pattern with respect to light reflected from the sensor.

The substrate may be functionalized with a polyelectrolyte 110 to adhere a metal 112 to the substrate 108. The metal may be a metal film or one or more layers of metal nanoparticles. In some cases, the metal includes discrete metal nanoparticles. In some embodiments, the dimensions of the metal layer (106) may range from about 1 nm to about 1000 nm thick with a variety of surface area to volume ratios. For example, in the LSPR application shown, polyelectrolyte 110 is used to adhere metal nanoparticles to the substrate 108. The polyelectrolyte 110 changes the charge at the surface of substrate 104, which allows the coupling of various metals to the sensor surface 104. The polyelectrolyte 110 can comprise, for example, a poly(allylamine hydrochloride) (PAH), a quaternary ammonium salt, an iminium salt or mixtures thereof. In other embodiments, positively charged polyelectrolytes may be used including quaternary ammonium polyelectrolytes. PAH useful in the practice of the present invention may be purchased from Sigma-Aldrich (MilliporeSigma, St. Louis, MO, USA).

In one embodiment, the metal 112 of sensor surface 104 comprises a plurality of nanoparticles forming a metal layer. In other embodiments, no polyelectrolyte is required and instead, a metal thin-film may be deposited directly onto substrate 108. The metal 112 together with the polyelectrolyte 110 and substrate 108 form the sensor surface 104 of the label-free sensor 102. Compositions of metal films and nanoparticles that can be used for SPR and LSPR include gold, silver, platinum, copper, gold coated silver, silver coated gold, combinations of these metals, and combinations of metal-coated nonmetal nanoparticles, and others. The size metal nanoparticles can vary from about 1 nm to about 1000 nm in various dimensions. The shape of the nanoparticles used can also vary. Useful nanoparticle shapes include but are not limited to, rods, stars, urchins, decahedra, hexagons, triangles, shells, prisms, platelets, spheres, rice, plates, cubes, cages, and bipyramids. Gold nanoparticles useful for the practice of the present invention may be purchased commercially from Sigma-Aldrich (MilliporeSigma, St. Louis, MO) or NanoComposix (San Diego, CA, USA).

The metal layer (112) may be coated with a chemical layer including a self-assembled monolayer (SAM) of linkers (106). For example, the chemical layer may include a compound of Formula I, to producing a linker-functionalized sensor surface:

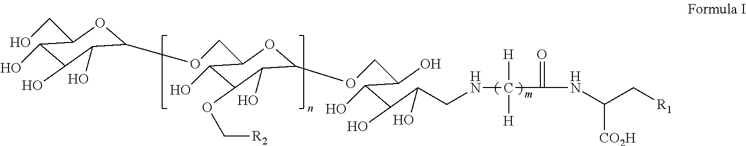

As shown in FIG. 2A, and in Formula I, the improved CMD linker may comprise at least one anchor 112 (R₁), at least one spacer 114, and at least one attachment group 116 comprising one or more attachment sites (R₂). In some cases, R₁ may include sulfur, carbon, nitrogen, oxygen, selenium, tellurium, or polonium. In some cases, R₂ may include carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, NHS-ester, nitrilotriacetic acid (NTA), azide, alkyne, a photocleavable moiety, or a photoreactive moiety. In some embodiments, “n” defines the number of repeat units in attachment group 116 which may be any whole-digit integer from 1 to 80 or from 1 to 30, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 repeat units. Still in some embodiments, “m” defines the number of repeat units in spacer 114 which may be any whole-digit integer from 1 to 6000. In some cases, m may be any whole digit integer from 1 to 3000. In some cases, m includes 1, 2, 3, 4, 5, 6, 7, 8, 9, 10, 11, 12, 13, 14, 15, 16, 17, 18, 18, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 repeat units. In some cases, m is 37. In some cases, n is 10.

In another embodiment, the chemical layer may include a compound of Formula II, to producing a linker-functionalized sensor surface:

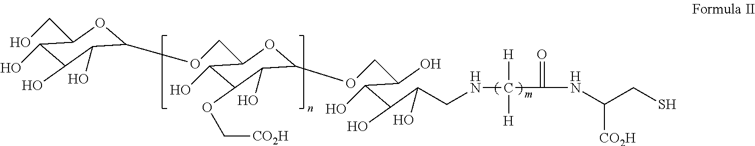

In some embodiments, “n” defines the number of repeat units in attachment group 116 which may be any whole-digit integer from 1 to 80 or from 1 to 30, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 repeat units. Still in some embodiments, “m” defines the number of repeat units in spacer 114 which may be any whole-digit integer from 1 to 6000. In some cases, m may be any whole digit integer from 1 to 3000. In some cases, m includes 1, 2, 3, 4, 5, 6, 7, 8, 9, 10, 11, 12, 13, 14, 15, 16, 17, 18, 18, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 repeat units. In some cases, m is 37. In some cases, n is 10.

One way to improve the signal of a small molecule analyte is to increase ligand immobilization onto the sensor by providing a plurality of attachment sites on the linker. This will increase the number of selective binding events of the analyte with the ligand and thus increase the signal strength produced by the label-free sensor. In the present application, the attachment group 118 is situated at a predetermined distance from the metal 112, and, in specific binding, the analyte of interest binds to the ligand distally (i.e., away from the metal 112). In non-specific binding (NSB), the analyte of interest may interact with other portions of the linker or metal, producing a false positive. Increasing the number of attachment sites per attachment group 118 may increase the ligand concentration, thereby reducing the opportunity for NSB while simultaneously increasing specific binding. This may occur through one of several mechanisms, namely, 1) there is an increase of negatively charged carboxyl groups (attachment sites) increases pre-concentration of the ligand (i.e., the concentration of ligand near attachment sites before ligand coupling) during immobilization; 2) self-assembly of the linker induced by the hydrophilic and hydrophobic portions may improve linker packing limiting mobility of the ligand to the metal 112; and 3) due to steric constraints created by the increased number of attachment sites, moving the attachment groups further away from the metal reduces additional steric burdens imposed on the analyte during binding allowing for the accurate measurement of the ligand and analyte interactions.

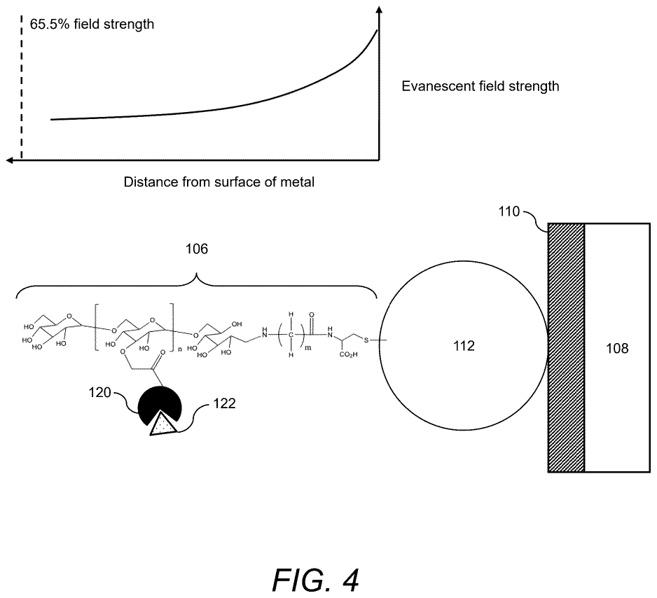

Referring now to FIG. 4, for a given sensor surface 104, a predetermined plasmon intensity (i.e., evanescent field strength) is provided. Ideally, the size of linker 106 is consistent with the plasmon intensity such that the majority of the analyte is located within 65.5% of the decay length of the surface plasmon. This may consequently increase or maximize the amount of peak shift caused by binding of the analyte with the ligand. In some cases, the decay length may be from about 5 nm to about 30 nm from the sensor surface 106. Accordingly, the length of linker 106 should be roughly proportional.

Disclosed herein is a method for functionalizing a sensor surface with a linker. The method includes cleaning the sensor surface to produce a cleaned sensor surface and exposing the cleaned sensor surface to a linker to produce a linker-functionalized sensor surface.

Cleaning the sensor surface may include removing contaminants from the sensor surface. As described in Example 2, cleaning the sensor surface may include removing organic contaminants. In some cases, cleaning the sensor surface includes rinsing the sensor surface with a solvent. In some cases, the solvent is water. In some cases, cleaning the sensor surface includes drying the sensor surface. In some embodiments, drying the sensor surface includes drying in ambient air. In some embodiments, drying the sensor surface includes drying with moving air. In some cases, the moving air may be produced by a fan. For example, an ion fan may be used.

The method further includes exposing the cleaned sensor surface to a to a solution comprising a linker to produce a linker-functionalized sensor surface. In some cases, the method includes exposing a solution comprising a compound of Formula I to a cleaned sensor surface to produce a linker-functionalized sensor surface:

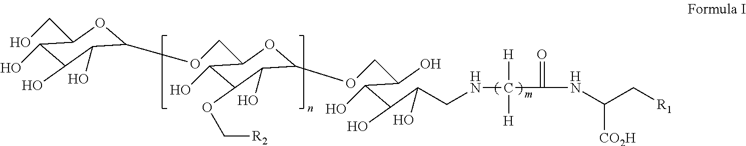

As shown in FIG. 2A, and in Formula I, the improved CMD linker may comprise at least one anchor 112 (R₁), at least one spacer 114, and at least one attachment group 116 comprising one or more attachment sites (R₂). In some cases, R₁ may include sulfur, carbon, nitrogen, oxygen, selenium, tellurium, or polonium. In some cases, R₂ may include carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, NHS-ester, nitrilotriacetic acid (NTA), azide, alkyne, a photocleavable moiety, or a photoreactive moiety. In some embodiments, “n” defines the number of repeat units in attachment group 116 which may be any whole-digit integer from 1 to 80 or from 1 to 30, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 repeat units. Still in some embodiments, “m” defines the number of repeat units in spacer 114 which may be any whole-digit integer from 1 to 6000. In some cases, m may be any whole digit integer from 1 to 3000. In some cases, m includes 1, 2, 3, 4, 5, 6, 7, 8, 9, 10, 11, 12, 13, 14, 15, 16, 17, 18, 18, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 repeat units. In some cases, m is 37. In some cases, n is 10.

In some cases, the method includes exposing a solution comprising a compound of Formula II to a cleaned sensor surface to produce a linker-functionalized sensor surface:

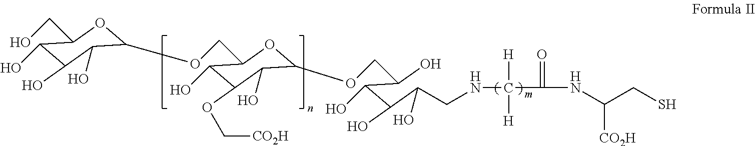

In some embodiments, “n” defines the number of repeat units in attachment group 116 which may be any whole-digit integer from 1 to 80 or from 1 to 30, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 repeat units. Still in some embodiments, “m” defines the number of repeat units in spacer 114 which may be any whole-digit integer from 1 to 6000. In some cases, m may be any whole digit integer from 1 to 3000. In some cases, m includes 1, 2, 3, 4, 5, 6, 7, 8, 9, 10, 11, 12, 13, 14, 15, 16, 17, 18, 18, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 repeat units. In some cases, m is 37. In some cases, n is 10.

Exposing the cleaned sensor surface to a solution comprising a linker may include a linker solution having a desirable linker concentration. In some cases, the linker concentration in the linker solution may be from about 0.1 mM to about 10 M, from about 0.1 mM to about 1 M, from about 0.1 mM to about 0.5 M. In some cases, the linker concentration in the linker solution may be about 0.1 mM, about 0.25 mM, about 0.5 mM, about 0.75 mM, about 1.0 mM, about 1.25 mM, about 1.5 mM, about 2.0 mM, about 2.5 mM, about 3.0 mM, about 3.5 mM, about 4.0 mM, about 4.5 mM, or about 5.0 mM linker. The linker solution may be an aqueous solution or a non-aqueous solution. In some cases, the non-aqueous solution may include a non-polar solvent. In some cases, the non-aqueous solution may include a polar solvent. In some cases, the linker solution may be an aqueous solution in Type 1 water.

Exposing the cleaned sensor surface to a solution comprising a linker may include an exposure time. In some cases, the exposure time is less than 1 minute, less than 5 minutes, less than 10 minutes, less than 15 minutes, less than 30 minutes, less than 60 minutes, less than 120 minutes, less than 180 minutes, less than 240 minutes, less than 300 minutes, less than 360 minutes, less than 420 minutes, or less than 480 minutes. In some cases, the exposure time is between 1 minute and 1440 minutes (24 hours). In some cases, the exposure time is between about 6 hours and about 36 hours, about 1 hour and about 48 hours, about 12 hours and about 24 hours, or about 10 hours and about 30 hours.

### Reagent Kits for Activating CMD-Based Label-Free Sensors

Reagent kits with required materials for setting up experiments may be provided to users with CMD-functionalized sensors. Reagent kits are designed and optimized to perform ideally with a specific linker. For example, the linker may include a compound of Formula:

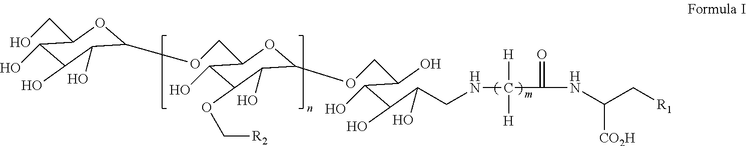

As shown in FIG. 2A, and in Formula I, the improved CMD linker may comprise at least one anchor 112 (R₁), at least one spacer 114, and at least one attachment group 116 comprising one or more attachment sites (R₂). In some cases, R₁ may include sulfur, carbon, nitrogen, oxygen, selenium, tellurium, or polonium. In some cases, R₂ may include carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, NHS-ester, nitrilotriacetic acid (NTA), azide, alkyne, a photocleavable moiety, or a photoreactive moiety. In some embodiments, “n” defines the number of repeat units in attachment group 116 which may be any whole-digit integer from 1 to 80 or from 1 to 30, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 repeat units. Still in some embodiments, “m” defines the number of repeat units in spacer 114 which may be any whole-digit integer from 1 to 6000. In some cases, m may be any whole digit integer from 1 to 3000. In some cases, m includes 1, 2, 3, 4, 5, 6, 7, 8, 9, 10, 11, 12, 13, 14, 15, 16, 17, 18, 18, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 repeat units. In some cases, m is 37. In some cases, n is 10.

In some cases, linker may include a compound of Formula II:

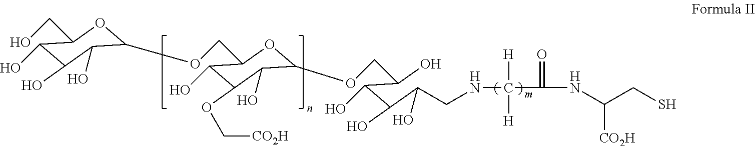

In some embodiments, “n” defines the number of repeat units in attachment group 116 which may be any whole-digit integer from 1 to 80 or from 1 to 30, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 repeat units. Still in some embodiments, “m” defines the number of repeat units in spacer 114 which may be any whole-digit integer from 1 to 6000. In some cases, m may be any whole digit integer from 1 to 3000. In some cases, m includes 1, 2, 3, 4, 5, 6, 7, 8, 9, 10, 11, 12, 13, 14, 15, 16, 17, 18, 18, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 repeat units. In some cases, m is 37. In some cases, n is 10.

These kits may include sensor preparation reagents, some combination of coupling reagents (i.e., EDC and NHS), ligand and/or capture molecule(s) (e.g. a ligand solution), immobilization buffer(s), blocking solution(s) and buffer solution(s). Additional components may be included in some circumstances, depending on the function of the reagent kit. A kit comprising a reagent system for coupling a ligand to the CMD-based linkers as presently described requires a specific concentration and ratio of coupling agent, for example, EDC/NHS, that may differ from other known label-free linkers, including CMD-based linkers. These concentrations vary with different linkers due to the total number and distribution of carboxyl groups available for reaction. The concentrations used for the presently described CMD linkers may be 200 millimolar (mM) EDC and 200 mM NHS, or a ratio of 1:1 EDC to NHS.

These concentrations are prescribed to maximize the potential surface capacity for ligand immobilization on the linker active sites. Generally, the higher the EDC/NHS concentrations, the more carboxy groups are activated and the lower the negative charge on the surface. Preconcentration is the process of immobilization in which ligand molecules are driven electrostatically to interact with the surface. This electrostatic interaction creates a higher local concentration of the ligand at the surface and must occur for the ligand to interact with the linker and become immobilized. Preconcentration is generally higher at lower EDC/NHS concentrations due to a higher negative charge of the surface interacting strongly with the positively charged ligand. Therefore, because more carboxyl groups are activated at higher concentrations, and electrostatic interactions with the ligand are greater at lower concentrations, there is an optimal concentration range where immobilization of the ligand is highest. The ratio of EDC:NHS is prescribed for optimal EDC-mediated crosslinking of the ligand to the activation sites on the CMD linker. This is similarly optimized based on maximum potential ligand immobilization and may vary between linker chemistries. When maximizing for ligand immobilization, there may be a point in which increasing immobilization further no longer allows for the same increase in subsequent binding and, therefore the analyte/ligand ratio suffers. Decreasing the EDC/NHS concentrations may reduce this effect. However, this can also introduce non-covalently bound ligands which is undesirable for accurate binding kinetics and multiple regeneration cycles. The EDC/NHS concentrations were chosen to minimize this non-covalent binding and maximize the potential surface capacity. The optimization of subsequent binding and the analyte/ligand ratio can be done by tuning the ligand concentration. In addition, because the number and distribution of carboxyl sites available for ligand binding on the presently described CMD linkers may differ from other label-free linkers, the theoretical ligand capacity of CMD and other said linkers may vary. This means that the concentration of ligand or capture molecule required to saturate the sensor surface may be different on a CMD-based linker than on other carboxyl-functionalized linkers. Depending on the function of the reagent kit, it may also be true that the desired ligand density is not saturation, but that which gives increased binding of subsequent biomolecules and optimal kinetic parameters. In this situation, the concentration of the ligand may likewise vary between CMD linkers and other label-free linkers. Accordingly, a reagent kit for the CMD linkers of the present disclosure may be provided with a different concentration of ligand or capture molecule in a ligand solution than an equivalent reagent kit for another label-free linker. In some instances, a reagent kit may be valid for more than one linker. In a similar vein, the difference in charge density and distribution of a linker can also modify how ligand molecules electrostatically interact with the surface. Because of this, ligands and/or capture molecules require a buffer of a specific pH to induce a charge to the ligand for optimal preconcentrating on the sensor surface. The optimal preconcentration condition is generally considered to be the pH at which preconcentration of the ligand is the highest and the rate of preconcentration is the fastest. Since the carboxyl groups are nearly always negatively charged, the pH must be a value that confers the ligand/capture molecule a positive charge, for an electrostatic interaction (i.e. preconcentration) to occur. In short, the pH is chosen to create a difference in charge between the ligand and the surface that is big enough to preconcentrate optimally. This pH is dependent on the charge distribution and density of the linker surface in combination with the isoelectric point of the ligand. For this reason, an immobilization buffer of a specific pH is generally prescribed for each reagent kit. The pH may be acidic, neutral, or basic. In some cases, the pH is about −1.0, about −0.5, about 0.0, about 0.5, about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5, about 10.0, about 10.5, about 11.0, about 11.5, about 12.0, about 12.5, about 13.0, about 13.5, or about 14.0.

### Methods

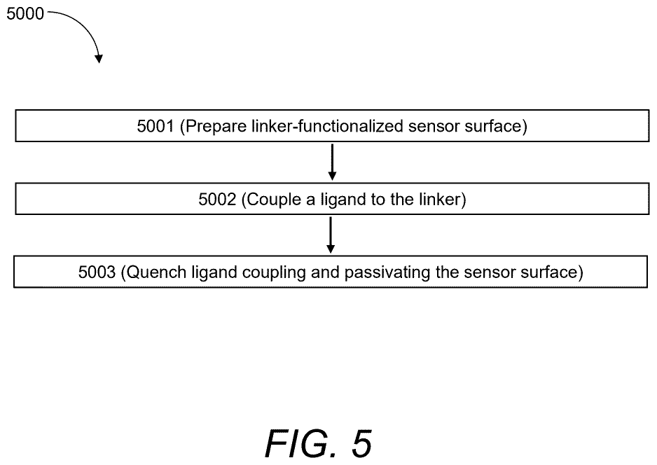

Referring now to FIG. 5, additionally disclosed herein are methods for using a reagent kit to immobilize a ligand on a sensor comprising a CMD-based linker (5000). An example of immobilizing a ligand on a sensor functionalized with a CMD-based linker is provided in Example 3, below. The method may include preparing the linker-functionalized sensor surface (5001), coupling a ligand to the linker (5002), and quenching the ligand coupling and passivating the sensor surface (5003).

The method includes a linker-functionalized sensor surface. In some cases, the linker-functionalized surface may be functionalized with a compound of Formula I:

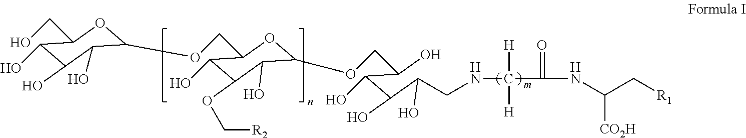

As shown in FIG. 2A, and in Formula I, the improved CMD linker may comprise at least one anchor 112 (R₁), at least one spacer 114, and at least one attachment group 116 comprising one or more attachment sites (R₂). In some cases, R₁ may include sulfur, carbon, nitrogen, oxygen, selenium, tellurium, or polonium. In some cases, R₂ may include carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, NHS-ester, nitrilotriacetic acid (NTA), azide, alkyne, a photocleavable moiety, or a photoreactive moiety. In some embodiments, “n” defines the number of repeat units in attachment group 116 which may be any whole-digit integer from 1 to 80 or from 1 to 30, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 repeat units. Still in some embodiments, “m” defines the number of repeat units in spacer 114 which may be any whole-digit integer from 1 to 6000. In some cases, m may be any whole digit integer from 1 to 3000. In some cases, m includes 1, 2, 3, 4, 5, 6, 7, 8, 9, 10, 11, 12, 13, 14, 15, 16, 17, 18, 18, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 repeat units. In some cases, m is 37. In some cases, n is 10.

In some cases, the linker-functionalized surface may be functionalized with a compound of Formula II:

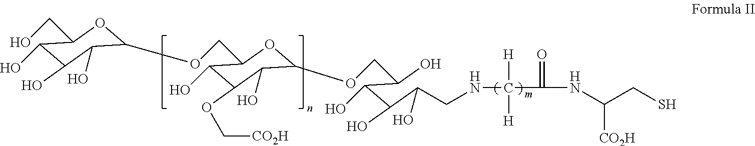

In some embodiments, “n” defines the number of repeat units in attachment group 116 which may be any whole-digit integer from 1 to 80 or from 1 to 30, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 repeat units. Still in some embodiments, “m” defines the number of repeat units in spacer 114 which may be any whole-digit integer from 1 to 6000. In some cases, m may be any whole digit integer from 1 to 3000. In some cases, m includes 1, 2, 3, 4, 5, 6, 7, 8, 9, 10, 11, 12, 13, 14, 15, 16, 17, 18, 18, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 repeat units. In some cases, m is 37. In some cases, n is 10.

The method may include preparing a linker-functionalized sensor surface (5001). Preparing a linker-functionalized sensor surface produces a cleaned linker-functionalized sensor surface. Preparing the linker-functionalized surface may include cleaning the linker-functionalized sensor surface. In some cases, cleaning the linker-functionalized sensor surface may include exposing the linker-functionalized sensor surface to one or more sensor preparation solutions.

In some cases, the sensor preparation solution may include an acid. In some cases, the sensor preparation solution may include an inorganic acid. In some cases, the inorganic acid is hydrochloric acid. In some cases, the inorganic acid may include H₂SO₄, H₃PO₄, HNO₃, HBr, HClO₄, or any combinations thereof. In some cases, the concentration of the acid is from about 0.1 mM to about 10 M, from about 0.1 mM to about 1 M, from about 0.1 mM to about 0.5 M, from about 0.1 mM to about 50 mM, from about 1.0 mM to about 50 mM, from about 5.0 mM to about 25 mM, from about 5.0 mM to about 15 mM, or from about 8.0 mM to about 12 mM. In some cases, the cleaning solution may include 10 mM acid. In some cases, the preparation solution may include a surfactant. In some cases, the surfactant is a non-ionic surfactant. In some cases, the surfactant includes Tween 20, Tween 21, Tween 40, Tween 60, Tween 80, Tween 85, Brij 30, Brij 58, Myrj 45, PEG-20 Laurate, or any combinations thereof. In some cases, the surfactant may be from about 0.01% to about 99%, from about 0.01% to about 50%, from about 0.01% to about 25%, from about 0.01% to about 10%, from about 0.01% to about 5.0%, from about 0.01% to about 1.0%, from about 0.05% to about 1.0%, from about 0.05% to about 0.75%, from about 0.05% to about 0.5%, or from about 0.05% to about 0.25%, based on the total volume of the sensor preparation solution. In some cases, the surfactant is about 0.1%, based on the total volume of the sensor preparation solution.

The method includes coupling a ligand to the linker of the cleaned linker-functionalized sensor surface (5002). Coupling a ligand to the linker of the cleaned linker-functionalized sensor surface produces a ligand-functionalized sensor surface. Suitable ligands include chemical moieties that may be coupled to an attachment site of the linker, and used to bind to an analyte. The ligand may be, for example, any binder, such as a protein, a peptide, an antibody, aptamer, polymer, DNA or other capture molecule having affinity for an analyte. In some cases, the ligand may be a solution. The ligand solution may be an aqueous solution or a non-aqueous solution. In some cases, the ligand solution may include additional components, including buffers. Coupling a ligand to the linker of the cleaned linker-functionalized sensor surface may include exposing the cleaned linker-functionalized sensor surface to a ligand solution. Exposing the cleaned linker-functionalized sensor surface to a ligand solution may be accomplished by submerging at least a portion of the cleaned linker-functional sensor surface in a ligand solution. In some cases, the ligand solution may be flowed or moved across the cleaned linker-functional sensor surface.

Exposing the cleaned linker-functionalized sensor surface to a ligand solution may include an exposure time. In some cases, the exposure time is less than 1 minute, less than 5 minutes, less than 10 minutes, less than 15 minutes, less than 30 minutes, less than 60 minutes, less than 120 minutes, less than 180 minutes, less than 240 minutes, less than 300 minutes, less than 360 minutes, less than 420 minutes, or less than 480 minutes. In some cases, the exposure time is between 1 minute and 1440 minutes (24 hours). In some cases, the exposure time is between 1 minute and 5 minutes, between 1 minute and 15 minutes, between 10 minutes and 15 minutes, between 1 minutes and 60 minutes, between 30 minutes and 60 minutes, between 1 minute and 120 minutes, or between 1 minute and 300 minutes.

Coupling a ligand to the linker of the linker-functionalized sensor surface can be accomplished with several known chemistries which result in one or more specific and/or covalent bonds forming between the linker and the ligand. In some cases, coupling a ligand to the linker of the linker-functionalized sensor surface includes a coupling agent which enables the binding of the ligand to the attachment sites of the linker. In some cases, 1-ethyl-3-(3-dimethylaminopropyl)carbodiimide (EDC of EDC/NHS) is used as a coupling agent. In this example, EDC is used to enable the covalent binding of a carboxyl group attachment site of the CMD-based linker's attachment group to an amine of a ligand using EDC/N-hydroxysuccinimide (NHS) coupling chemistry. In some cases, EDC/sulfo-NHS or 4-(4,6-Dimethoxy-1,3,5-triazin-2-yl)-4-methylmorpholinium chloride (DMTMM) can be used as a coupling agent.

The method further includes quenching the coupling of a ligand to the linker of the cleaned linker-functionalized sensor surface (5003). Quenching the coupling of a ligand to the linker of the cleaned linker-functionalized sensor surface produces a functionalized-passivated sensor surface. Quenching the coupling of a ligand to the linker of the cleaned linker-functionalized sensor surface refers to, after the initial reaction of the ligand with attachment sites on the linker, stopping or preventing further reactions between the unreacted attachment sites of the linker. In some cases, quenching is accomplished by exposing the ligand-functionalized sensor surface to one or more components selected to react with the unreacted attachment sites. In some cases, the ligand-functionalized sensor surface is exposed to a blocking solution, which refers to a solution including one or more components selected to prevent or block further reactions of unreacted attachment sites of the linker. In some cases, the one or more components of the blocking solution may include one or more amines. In some case, the one or more amines is a primary amine. In some cases, the blocking solution may include ethanolamine, tris(hydroxymethyl)aminomethane (tris), glycine, lysine, amino alcohols (e.g., 1-amino-2-propanol), PEG-amine derivatives, proteins, or any combinations thereof. In some cases, the amine has a concentration from about 0.1 M to about 10 M, from about 0.2 M to about 8 M, from about 0.5 M to about 4 M, from about 0.8 M to about 2 M, from about 0.8 M to about 1.5 M, or from about 0.9 M to about 1.2 M. In some cases, the amine is about 1.0 M. In some cases, the blocking solution includes a surfactant. In some cases, the surfactant is a non-ionic surfactant. In some cases, the surfactant includes Tween 20, Tween 21, Tween 40, Tween 60, Tween 80, Tween 85, Brij 30, Brij 58, Myrj 45, PEG-20 Laurate, or any combinations thereof. In some cases, the surfactant may be from about 0.01% to about 99%, from about 0.01% to about 50%, from about 0.01% to about 25%, from about 0.01% to about 10%, from about 0.01% to about 5.0%, from about 0.01% to about 1.0%, from about 0.05% to about 1.0%, from about 0.05% to about 0.75%, from about 0.05% to about 0.5%, or from about 0.05% to about 0.25%, based on the total volume of the sensor preparation solution. In some cases, the surfactant is about 0.1%, based on the total volume of the sensor preparation solution. In some cases, the blocking solution includes a surfactant and a primary amine. In some cases, when the attachment site includes a carboxyl group and the blocking solution includes a primary amine, the primary amine reacts with the unreacted carboxyl groups (i.e., carboxyl groups which did not react with a ligand). In some cases, the method optionally further includes exposing the quenched ligand-functionalized sensor surface to a sensor preparation solution comprising an acid and a surfactant, as described above. Exposing the quenched ligand-functionalized sensor surface to a sensor preparation solution produces a cleaned quenched ligand-functionalized sensor surface.

In some cases, the quenching of the coupling of a ligand to the linker of the cleaned linker-functionalized sensor surface additionally passivates the surface of the sensor. In some cases, quenching the coupling of a ligand to the linker of the cleaned linker-functionalized sensor surface and passivating the sensor surface may be performed in a single step. In some cases, quenching the coupling of a ligand to the linker of the cleaned linker-functionalized sensor surface and passivating the sensor surface may be performed in multiple steps. In one case, the blocking solution may quench the coupling of a ligand to the linker of the cleaned linker-functionalized sensor surface and may additionally remove ligand that is non-specifically interacting with the linker. For example, the blocking solution may be used to deactivate unreacted NHS-esters and help to remove any excess ligand that is electrostatically bound to the linker. In some cases, bovine serum albumin (BSA) may be used for sensor surface passivation.

In some cases, quenching the coupling of a ligand to the linker of the cleaned linker-functionalized sensor surface (5003) optionally further includes cleaning the functionalized-passivated sensor surface. In some cases, cleaning the functionalized-passivated sensor surface may include exposing the functionalized-passivated sensor surface to one or more cleaning solutions. In some cases, the cleaning solution may include an acid. In some cases, the cleaning solutions may include an inorganic acid. In some cases, the inorganic acid is hydrochloric acid. In some cases, the inorganic acid may include H₂SO₄, H₃PO₄, HNO₃, HBr, HClO₄, or any combinations thereof. In some cases, the concentration of the acid is from about 0.1 mM to about 10 M, from about 0.1 mM to about 1 M, from about 0.1 mM to about 0.5 M, from about 0.1 mM to about 50 mM, from about 1.0 mM to about 50 mM, from about 5.0 mM to about 25 mM, from about 5.0 mM to about 15 mM, or from about 8.0 mM to about 12 mM. In some cases, the cleaning solution may include 10 mM acid. In some cases, the preparation solution may include a surfactant. In some cases, the surfactant is a non-ionic surfactant. In some cases, the surfactant includes Tween 20, Tween 21, Tween 40, Tween 60, Tween 80, Tween 85, Brij 30, Brij 58, Myrj 45, PEG-20 Laurate, or any combinations thereof. In some cases, the surfactant may be from about 0.01% to about 99%, from about 0.01% to about 50%, from about 0.01% to about 25%, from about 0.01% to about 10%, from about 0.01% to about 5.0%, from about 0.01% to about 1.0%, from about 0.05% to about 1.0%, from about 0.05% to about 0.75%, from about 0.05% to about 0.5%, or from about 0.05% to about 0.25%, based on the total volume of the cleaning solution. In some cases, the surfactant is about 0.1%, based on the total volume of the cleaning solution.

The method may further include equilibration of the sensor surface. Equilibration of the sensor surface may occur before or after any stage of the method. In some cases, equilibration may occur before or after preparing a linker-functionalized sensor surface (5001). In some cases, equilibration may occur before or after coupling a ligand to the linker of the cleaned linker-functionalized sensor surface (5002). In some cases, equilibration may occur before or after quenching the coupling of a ligand to the linker of the cleaned linker-functionalized sensor surface (5003). Equilibration of the sensor surface may be accomplished by exposing the sensor surface to one or more buffers. In some cases, the buffer may be acidic, basic, or neutral. In some cases, the buffers may include PBS-T (phosphate-buffered saline with 0.1% Tween 20), PBS (phosphate-buffered saline), MES (2-morpholinoethanesulfonic acid), acetate, 4-(2-hydroxyethyl)-1-piperazineethanesulfonic acid (HEPES), citrate, formate buffers, or any combinations thereof. In some cases, the buffer solution may be from about 0.01% to about 99%, from about 0.01% to about 50%, from about 0.01% to about 25%, from about 0.01% to about 10%, from about 0.01% to about 5.0%, from about 0.01% to about 1.0%, from about 0.05% to about 1.0%, from about 0.05% to about 0.75%, from about 0.05% to about 0.5%, or from about 0.05% to about 0.25%, based on the total volume of the buffer solution.

### Additional Compounds

Also disclosed herein are compounds including a first monomer including compound of Formula III:

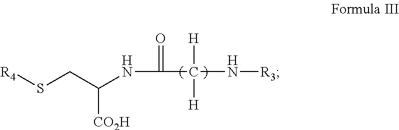

where R₃ includes a monosaccharide, a disaccharide, a polysaccharide, or an oligosaccharide; R₄ includes hydrogen or a protecting group. In some cases, the protecting group is triphenylmethyl.

In some embodiments, “m” defines the number of repeat units in spacer 114 which may be any whole-digit integer from 1 to 6000. In some cases, m may be any whole digit integer from 1 to 3000. In some cases, m includes 1, 2, 3, 4, 5, 6, 7, 8, 9, 10, 11, 12, 13, 14, 15, 16, 17, 18, 18, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 repeat units. In some cases, m is 37. In some cases, n is 10.

In some cases, the R₃ includes a monosaccharide can include triose, pentose, hexose, heptose, or any combinations thereof. In some cases, the monosaccharide includes glucose, fructose, galactose, ribose, deoxyribose, mannose, and any combinations thereof.

In some cases, the R₃ includes a disaccharide. In some cases, the disaccharide is sucrose, lactose, maltose, trehalose, cellobiose, chitobiose, kojibiose, nigerose, isomaltose, sophorose, laminaribiose, gentiobiose, turanose, maltulose, leucrose, isomaltulose, gentiobiulose, mannobiose, melibiose, allolactose, lactulose, rutinose, rutinulose, xylobiose, or any combinations thereof.

In some cases, the R₃ includes a polysaccharide. In some cases, the polysaccharide includes dextran, amylose, glycogen, cellulose, chitin, starch, pectin, hemicellulose, inulin, agar, alginate, or any combinations thereof.

In some cases, the R₃ includes an oligosaccharide. In some cases, the oligosaccharide includes fructo-oligosaccharides, galactooligosaccharides, mannan oligosaccharides, or any combinations thereof.

In some embodiments, the R₃ includes a compound of Formula IV:

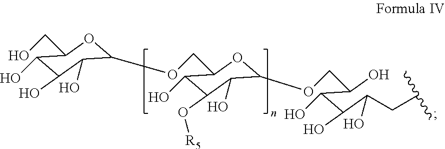

where R₅ includes H or carboxyl. In some embodiments, “n” defines the number of repeat units in attachment group 116 which may be any whole-digit integer from 1 to 80 or from 1 to 30, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 repeat units.

### EXAMPLES

The following illustrative examples are representative of embodiments of the software applications, systems, and methods described herein and are not meant to be limiting in any way.

### Example 1—Synthesis of CMD-Based Linker

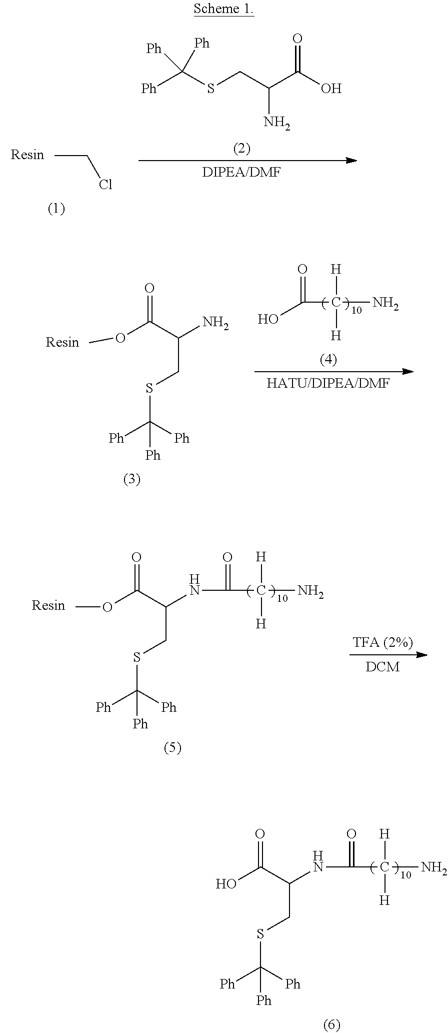

In one example a CMD-based linker was synthesized. Scheme 1 shows the steps for synthesizing SAM fraction (6) of the CMD-based linker via solid-phase peptide synthesis (SPPS).

First, a commercially available Fmoc-cysteine derivative (2) was loaded on the solid 2-Cl-trityl resin (1) by shaking for 4 hours. 5.0 g unloaded 2-chlorotrityl resin was transferred to a 100 ml glass reactor with 50 mL DCM and soaked for 30 minutes, followed by discharge of the DCM using a vacuum line. A cocktail of 8.8 g Fmoc-Cys (2) was dissolved in 20 mL dry DMF and 5.3 mL DIPEA (6× ratio), filtered, and then added to the reactor containing the resin. The reactor was purged with nitrogen and shaken for 4 hr. The solvent was then discharged under vacuum, followed by washing with DMF three times, and DCM five times. A solution of 8.5:1:0.5 DCM:MeOH:DIPEA was then prepared and added to the reactor, followed by shaking for 1 hour at room temperature. The resin was then rinsed three times with DCM, three times alternating DCM and MeOH, then five times with DCM and dried under vacuum. To remove the Fmoc group for nitrogen deprotection 50 mL 20% piperidine in DMF was added to the reactor followed by shaking for about 20 min. After shaking, the piperidine solution was removed from the reactor under vacuum. This deprotection step was repeated two more times, followed by rinsing of the resin three times with DMF, three times alternating DCM and MeOH, then three times DCM, followed by drying under vacuum for about 5 min.

To add the hydrophobic chain to the resin-bound cysteine, in this example, 6.353 g Fmoc-11-amino-undecylic acid in a 3× ratio with 6.083 g HATU in a 3.2× ratio in 30 mL dry DMF. 7.839 mL DIPEA was then added to the reactor and the mixture shaken overnight at room temperature. The resin was then rinsed three times with DMF, three times alternating DCM and MeOH, then five times with DCM and dried over vacuum for about 5 minutes. It is understood that the same general procedure may be used for a variety of hydrocarbon chain lengths and types to produce a variety of linkers, wherein the hydrophobic chain has a free amino group for later functionalization.

To remove the Fmoc protecting group, 50 mL 20% piperidine in DMF was added to the reactor and the mixture shaken for about 20 minutes. After shaking, the piperidine solution was drained from the reactor under vacuum. The resin was then rinsed three times with DMF, three times alternating DCM and MeOH, then five times with DCM followed by drying over vacuum.

To cleave the linker from the resin a solution of 2% TFA in DCM was added followed by shaking at room temperature for 30 min. After shaking, the cleavage solution was drained from the reactor and collected. The cleavage reaction was then done an additional two times (3× total cleavage steps), using a fresh 50 mL of cleavage solution each time. After the third round of cleavage, rinse the resin with another 10-20 mL of fresh cleavage solution and drain directly into a clean dry falcon tube. To the collected cleavage solution was added about 200 mL diethyl ether and 20-30 mL methanol to the flask to aid TFA evaporation, and the cleavage solution, solvent, and product was connected to a rotary evaporator to remove the solvent, yielding a brown oil. The oil was dissolved in a minimum amount of acetone (<8 mL) and then the solution was transferred to a 20 ml glass scintillation vial. The vial was connected to a rotary evaporator to remove as much solvent as possible and this step was repeated two more times. The acetone solutions were combined, vacuum dried, and the product could then be stored in a freezer for future use. The product of this solid phase synthesis is the trityl protected sulfur anchor group attached to an amino-hydrocarbon chain. This intermediate can then be functionalized with a cellulosic capable of being carboxymethylated for coupling with a ligand. Alternatively, this intermediate can be used as an anchor group for an SPR sensor linker and functionalized after anchoring to a metal sensor layer.

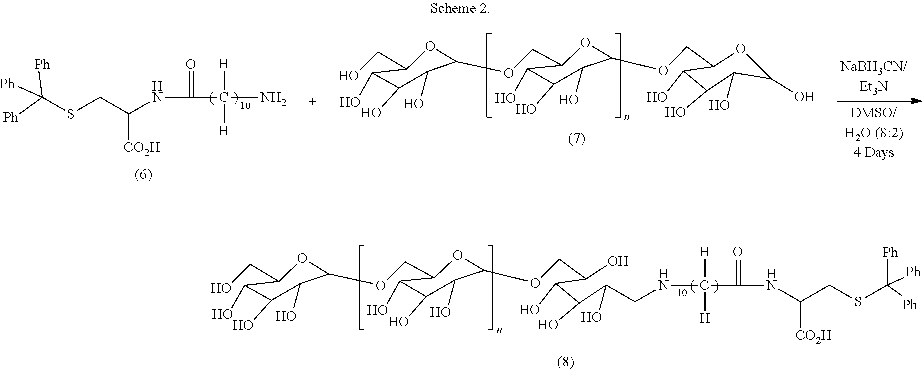

Scheme 2 shows the steps for coupling SAM fraction (6) to dextran via reductive amination.

Reductive amination of the protected anchor-amino-hydrocarbon with dextran provides dextran end-functionalization and formation of the SPR linker for ligand crosslinking. For this reaction a dextran with MW of 6000 g/mol (7) was used however, other dextran with lower or higher molecular weights are also feasible to be incorporated. Based on the decay length of the gold nanoparticles used on the LSPR sensor, this dextran was selected to provide a linker that provides an average of 27 nm in linear length. Dextran is used herein as an example polysaccharide, however it is understood that other polysaccharides capable of carboxymethylation may also be used. The dextran may be of variable length and molecular weight, varying from, for example, from 0.5 to 2000 kilodaltons.

Reductive amination of the primary amine on the functionalized aminohydrocarbon (6) with the dextran in the presence of a base and a reducing agent provides a specific regiochemistry wherein the reducible terminal glucose ring (anomeric glucose) will be irreversibly opened providing a single site of functionalization on the dextran chain. Although this reaction is very slow and takes at least four days to be completed, the mechanism of this reaction guarantees the coupling reaction to exclusively at a terminal glucose unit in the structure of dextran. During reductive amination, the thiol group on the anchor remains protected to prevent its adverse effects on the reductive amination reaction. The thiol protecting group can then be removed as the last step of the synthesis protocol.

In a 500 mL round bottom flask 5.0 g dextran (7) (MW 6000 g/mol, 0.83 mmol) was dissolved in 25 mL Ultrapure, Type 1 water to which was added 50 mL DMSO and the mixture was stirred magnetically at 500 rpm. Ultrapure, Type I water is defined by the American Society for Testing and Materials (ASTM) as having a resistivity of >18 MΩ-cm, a conductivity of <0.056 μS/cm, and <50 ppb of Total Organic Carbons (TOC). 4.55 g of the anchor-functionalized hydrocarbon (6) (MW: 546 g/mol, 10 eq) was dissolved in 25 mL DMSO and added to the reactor slowly. 2.9 mL triethylamine (M:101 g/mol, d=0.726 g/mL, 25 eq) was then added to the reactor followed by 3.9 g sodium cyano-borohydride (M:63 g/mol, 75 eq) dissolved in 25 mL. The reaction medium was a mixture of DMSO/water (8:2) and the ratio is especially important as in this reaction two components are reacting that are opposite in their hydrophilicity natures. Dextran is a hydrophilic component and the anchor functionalized hydrocarbon has a very hydrophobic nature, so a mixture of DMSO/water is needed to keep them in solution during the long reaction time. The solution was stirred for four days at 60° C. and then continued for two days at room temperature under a nitrogen atmosphere.

The reaction was completed in four days and the material was purified before proceeding to the next step via crystallization from acetone three times. After four days of stirring, the product was precipitated in 400 mL acetone while stirring vigorously. The precipitation was separated by centrifuging and at 4000 rpm for two minutes. This process was repeated two more times using fresh acetone to provide a pure white powder of end-functionalized dextran (8). The product was washed with ether, vacuum-dried, and stored at −20° C. It was confirmed that the dextran ring opens as a result of the coupling to the hydrophobic portion to the dextran.

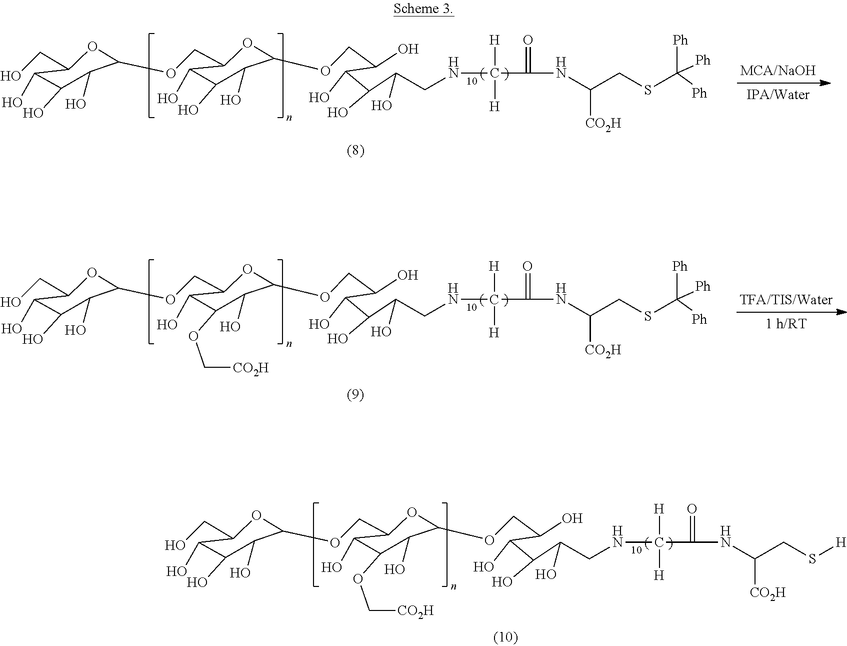

Scheme 3 shows the steps for carboxylation of end-functionalized dextran (8).

A carboxylation reaction is generally a heterogeneous solid/liquid reaction and suffers from scalability. To address the scalability issue, end-functionalized dextran (8) was carboxylated (9) in a semi-emulsion system. End-functionalization can be achieved by dissolving dextran and base in water and reaction with monochloroacetic acid (MCA) in isopropanol (IPA). Through this reaction, carboxyl groups can be added to the dextran structure. To increase the carboxylation level the reaction can also be repeated. It is noted that increasing the reaction time or temperature did not lead to a higher carboxylation level. Optimization of the level of carboxylation on the surface can be done via a standard sensor method in which recombinant protein A was immobilized as a ligand and IgG as the analyte.

For carboxylation of the anchor-hydrocarbon functionalized dextran (8), 5.0 g end-functionalized dextran (8) was dissolved in 31.25 mL Type 1 water. 5.56 g NaOH (MW 40 g/mol, 5 eq of glucose units) was added to dissolve. In a 250 mL round bottom flask, as a reactor, 6.17 g mono-chloroacetic acid (MW 94.5, 0.47 eq of NaOH) was dissolved in 125 mL isopropanol (IPA) using a magnetic stir bar at RT and 700 rpm. The solution of functionalized dextran (8) was added to the reactor and the reactor was transferred to a pre-heated oil bath at 60° C. to stir for two hours. After two hours, the reaction was removed from the oil bath to cool down to RT and then 1 mL of acetic acid was added and stirred for 10 min. The pH after 10 min should be between 5-6. After reaching a pH of 5-6 the stirrer was turned off to let the IPA phase be separated from the water phase. The IPA phase was then removed, and the aqueous phase was precipitated dropwise in 400 mL of methanol.

The precipitate was washed with methanol one more time and was then washed with acetone and then ether and then dried under vacuum. The carboxylation process was repeated one more time from the beginning to reach the desired carboxylation level.

After the carboxylation, the thiol-protecting groups were removed simply by dissolving materials in a mixture of TFA/TIS/water (95:2.5:2.5). Final precipitation and purification were done in diethyl ether followed by vacuum drying of the final linker (10) and storing it at −80° C. The characterization of the final linker (10) was based on HPLC-MS.

### Example 2—Sensor Surfacing with CMD-Based Linker

In one example of functionalizing a gold nanoparticle LSPR sensor with the presently described linker, the surface comprising gold nanoparticles are first cleaned to ensure that organics are removed. To do this the surface is rinsed thoroughly with Type 1 water. The gold nanoparticle surface was then dried thoroughly with an ion fan. A solution comprising 1.0 mM linker (10) as presently described was prepared in Type 1 Water. The solution was sonicated until clear and colorless, and was then filtered with a 0.2 micrometer (m) syringe filter. The metal sensor surface was then incubated in the linker solution for 12-24 hours at room temperature to attach the linker to the metal on the sensor surface.

### Example 3—Ligand Immobilization on Sensor

Prior to the activation of a label-free sensor surface comprising a CMD-based linker, one or more sensor preparation solutions are used to clean the sensor surface. The prescribed sensor preparation solution for the presently disclosed sensor comprising a CMD-based linker includes 10 mM HCl with 0.1% Tween 20. After preparation, a buffer solution comprising a ligand (i.e., a ligand solution) is introduced to the sensor surface. In some cases, EDC is used as a coupling agent to covalently attach a carboxyl group attachment site of the CMD-based linker's attachment group to an amine of the ligand using EDC/NHS coupling chemistry. After the reaction of the carboxyl groups with the ligand, the remaining unreacted carboxyl groups are quenched by adding a blocking solution. A blocking solution is typically a small molecule with a primary amine group capable of reacting with the unreacted carboxyl groups without significantly alternating the signal generated by the label-free sensor. The blocking solution is intended to prevent molecules in downstream steps from being immobilized on the linker in a non-specific manner. In addition, the blocking solution may be used to deactivate unreacted NHS-esters and help to remove any excess ligand that is electrostatically bound to the linker. In the present example, the blocking solution is 1M ethanolamine+0.1% Tween 20, however, other blocking solutions may be used. Between each step of the immobilization process, a buffer may be used to equilibrate the sensor surface, such as a PBS-T (Phosphate-buffered saline with 0.1% Tween 20) buffer. However, other buffers may be used.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the invention shall also cover any such alternatives, modifications, variations or equivalents. It is intended that the following claims define the scope of the disclosure and that methods and structures within the scope of these claims and their equivalents be covered thereby.

What is claimed is:

1. A composition for label-free molecular analysis, comprising a compound of Formula I: 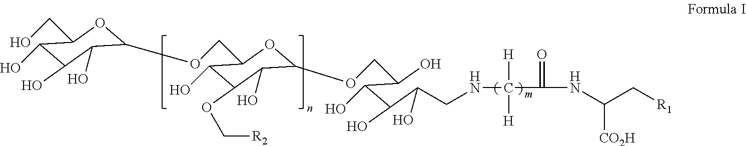
wherein R₁ comprises sulfur, carbon, nitrogen, oxygen, selenium, tellurium, or polonium;
wherein R₂ comprises carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, N-hydroxysuccinimide ester, nitrilotriacetic acid, azide, alkyne, a photocleavable moiety, or a photoreactive moiety;
wherein m is about 1 to about 6000; and
wherein n is about 1 to about 80.

2. The composition of claim 1, wherein R₁ comprises sulfur and is selected from the group consisting of thiol, sulfide, disulfide, or any combinations thereof.

3. The composition of claim 1, wherein R₂ comprises carboxyl.

4. The composition of claim 1, wherein m is about 1 to about 3000.

5. The composition of claim 1, wherein n is about 1 to about 30.

6. A sensor for label-free molecular analysis, comprising:
a sensor surface comprising a substrate coated with a metal layer; and
a linker disposed on the metal layer comprising a compound of Formula I: 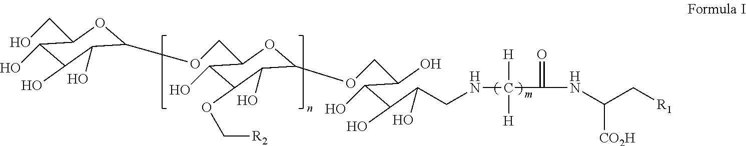
wherein comprises sulfur, carbon, nitrogen, oxygen, selenium, tellurium, or polonium,
wherein R₂ comprises carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, N-hydroxysuccinimide ester, nitrilotriacetic acid, azide, alkyne, a photocleavable moiety, or a photoreactive moiety,
wherein m is about 1 to about 6000, and
wherein n is about 1 to about 80.

7. The sensor of claim 6, wherein metal layer comprises a plurality of nanoparticles.

8. The sensor of claim 6, wherein the substrate comprises an optical fiber.

9. The sensor of claim 6, wherein the sensor is a localized surface plasmon resonance (LSPR) sensor.

10. The sensor of claim 6, wherein the metal layer comprises gold nanoparticles; and wherein the linker comprises a compound of Formula II: 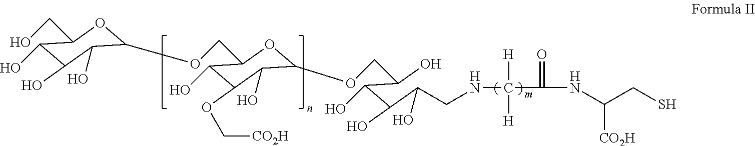
wherein m is 1 to about 6000, and
wherein n is 1 to about 80.

11. A reagent kit for treating a sensor surface, the sensor surface comprising a linker comprising a compound of Formula I: 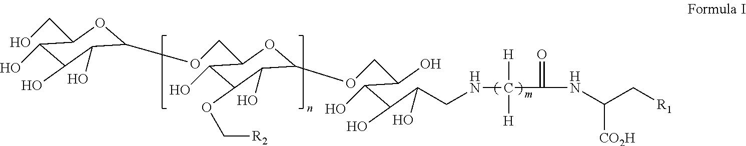
wherein R₁ comprises sulfur, carbon, nitrogen, oxygen, selenium, tellurium, or polonium,
wherein R₂ comprises carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, N-hydroxysuccinimide ester, nitrilotriacetic acid, azide, alkyne, a photocleavable moiety, or a photoreactive moiety,
wherein m is about 1 to about 6000, and
wherein n is about 1 to about 80,
the reagent kit comprising:
a first solution comprising an acid and a surfactant;
a second solution comprising a ligand; and
a third solution comprising an amine.

12. The reagent kit of claim 11, wherein the linker comprises a compound of Formula II: 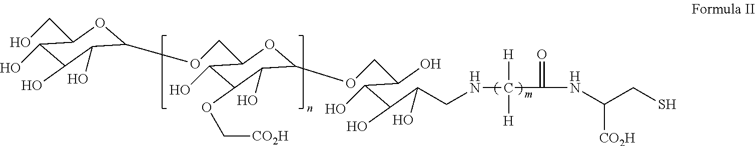
wherein m is 1 to about 6000, and
wherein n is 1 to about 80.

13. The reagent kit of claim 11, wherein the first solution comprises an inorganic acid and a surfactant.

14. The reagent kit of claim 11, wherein the second solution comprises a protein, a peptide, an antibody, aptamer, polymer, DNA, or any combinations thereof.

15. The reagent kit of claim 11, wherein the third solution comprises a primary amine.

16. A composition for label-free molecular analysis comprising a compound of Formula III: 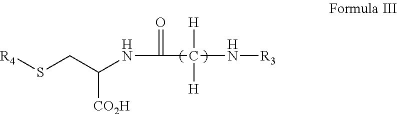
wherein R₃ comprises a monosaccharide, a disaccharide, a polysaccharide, or an oligosaccharide,
wherein R₄ comprises H or a protecting group,
wherein m is about 1 to about 6000.

17. The composition of claim 16, wherein R₃ comprises a polysaccharide, and wherein the polysaccharide is selected from the group consisting of dextran, amylose, glycogen, cellulose, chitin, starch, pectin, hemicellulose, inulin, agar, alginate, or any combinations thereof.

18. The composition of claim 16, wherein R₃ comprises a compound of Formula IV: 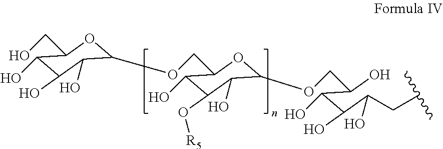
wherein n is about 1 to about 80; and
R₅ comprises hydrogen or carboxyl.

19. A method for using a reagent kit to immobilize a ligand on a linker-functionalized sensor surface comprising a compound of Formula I: 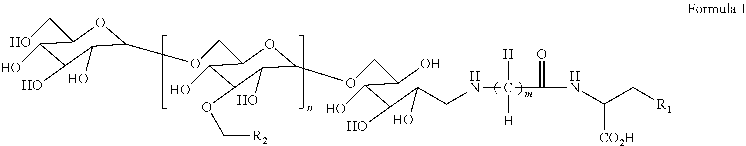
wherein R₁ comprises sulfur, carbon, nitrogen, oxygen, selenium, tellurium, or polonium,
wherein R₂ comprises carboxyl, biotin, streptavidin, neutravidin, avidin, hydrazine, aminooxy, thiol, epoxy, tetrazine, trans-cyclooctene, aryl azide, diazirine, polyethylene glycol, zwitterionic moieties, amine, aldehyde, pyridyl-disulfide, maleimide, N-hydroxysuccinimide ester, nitrilotriacetic acid, azide, alkyne, a photocleavable moiety, or a photoreactive moiety,
wherein m is about 1 to about 6000, and
wherein n is about 1 to about 80;
the method comprising:
exposing the linker-functionalized sensor surface to a sensor preparation solution comprising an acid and a surfactant to produce a cleaned linker-functionalized sensor surface,
exposing the cleaned linker-functionalized sensor surface to a ligand solution comprising a ligand to produce a ligand-functionalized sensor surface; and
exposing the ligand-functionalized sensor surface to a blocking solution comprising an amine to produce a quenched ligand-functionalized sensor surface.

20. The method of claim 19, wherein the linker-functionalized sensor surface comprises a compound of Formula II: 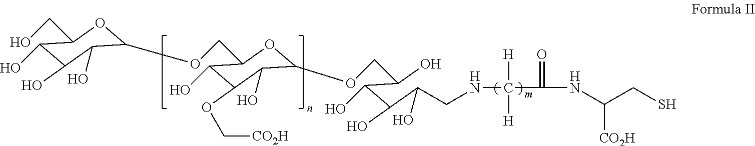
wherein m is 1 to about 6000, and
wherein n is 1 to about 80.
